(19) United States
(12) Patent Application Publication
(10) Pub. No.: US 2025/0001030 A1
(43) Pub. Date: Jan. 2, 2025

(54) DISINFECTION ROBOTS AND DISINFECTION METHODS

(71) Applicants: Johnson Yiu-Nam LAU, Houston, TX (US); Avalon Steritech Limited, Hong Kong (CN)

(72) Inventors: Johnson Yiu-Nam LAU, Houston, TX (US); Wai Hong HO, Hong Kong (CN)

(73) Assignees: Johnson Yiu-Nam LAU, Houston, TX (US); Avalon Steritech Limited, Hong Kong (CN)

(21) Appl. No.: 18/764,249

(22) Filed: Jul. 4, 2024

Related U.S. Application Data
(63) Continuation of application No. PCT/US2023/010068, filed on Jan. 3, 2023.
(60) Provisional application No. 63/296,433, filed on Jan. 4, 2022.
(60) Provisional application No. 63/307,123, filed on Feb. 6, 2022.

(51) Int. Cl. A61L 2/22; A61L 2/24

(52) U.S. Cl. CPC A61L 2/22; A61L 2/24; A61L 2202/14; A61L 2202/15; A61L 2202/16

(57) ABSTRACT

A cleaning and disinfection system. More particularly, aspects of the invention relate to devices, systems and methods for cleaning and/or disinfection of spaces by robotic means. In one aspect, the present invention provides disinfection robots that may be conveniently deployed at target spaces including public transits, are able to navigate through small and narrow spaces and disinfect such spaces effectively and efficiently. In another aspect, the present invention provides an atomization system the is capable of generating droplets of at least 40 microns in size and of substantially the same size. In yet another aspect, the present invention provides methods for disinfecting a confined space such as a space on a railway system or an aircraft, or a confined room using droplets of a disinfection composition.

### CROSS-REFERENCE TO RELATED APPLICATIONS

This application is a continuation of International Patent Application No. PCT/US2023/010068, filed Jan. 3, 2023, which also claims priority to a provisional application Ser. No. 63/296,433, filed on 4 Jan. 2022, and a provisional application Ser. No. 63/307,123, filed on 6 Feb. 2022, whose disclosures are incorporated by reference in their entirely herein.

### FIELD OF INVENTION

This invention generally relates to a disinfection system and a disinfection method. More particularly, a disinfection mobile robot that may be conveniently deployed for cleaning, disinfecting and sterilizing public space in mass transportation, for example, aircraft cabin and railway system. In particular, aspects of the invention relate to a system and method for controlling mobile robots in cleaning, disinfecting and sterilizing public space.

### BACKGROUND

Millions of people are using public transits every day. Therefore, it is essential to keeping the public space of the public transits clean. The public transits service providers usually perform one major cleaning at once a while and minor cleaning a couple of times per day. Cleanings, which generally refer to the removal of visible foreign matters such as dirt, dust and crumbs from surfaces or objects, are usually carried out manually and the cleaning equipment used (e.g. vacuum cleaner, mop and sweeper) is mainly specialized in removing visible foreign matter (e.g. dust and trash) only.

After the beginning of the COVID-19 pandemic, the public confidence in public transportation drops significantly due to hygiene concerns. The government also concerns public transits may increase the spreading of COVID-19 or other viruses in the community.

The concerns of risks of infections during air travel is particularly immense. It was speculated that the rate or risk of viral transmission on flights is higher than those via other mode of public transits (e.g. trains and buses) because passengers are usually packed into a confined space in the aircraft cabin and the flight times are much longer than the duration of typically train or bus rides and may be up to 15 hours or more.

In view of that, the public transits service providers begin to disinfect the public area to reduce the risk of spreading disease in public transits.

As of the date of this invention, disinfection operations in the public transits are mostly carried out manually. Manual disinfection has a number of disadvantages such as inconsistency in the degree of disinfection, overuse of disinfectants, increased exposure to pathogens by cleaning operators and thereby increasing their and the community's risks of infection, increased amount of waste caused by uses of disposable protective gears of cleaning operators, increased time, cost and employees' injuries, and ineffective disinfection at areas that are hard to be reached by human.

As an alternative or supplement to manual cleaning and/or disinfection, robotic cleaning and/or disinfection systems have been developed. The robotic approach offers many benefits over the traditional manual approach, including but not limited to, increasing in consistency and reliability, decreasing in risks of infection, reduction of the amount of waste, and reducing the time, cost, labor demand as well as employees' injuries. Consequently, the environment, social, and governance (ESG) ratings of companies performing cleaning and/or disinfection using robotic systems improves compared to those using traditional manual techniques.

Cleaning and/or disinfection robots may be deployed at a variety of places, indoor or outdoor. Yet, some of the places are narrow and small and some of them may have a lot of obstacles. Further, different places may have different flooring materials (e.g. carpet, plastic, wood, metal, stone, etc.) and some may have uneven or inclined surfaces. For example, train interiors may include different flooring materials and uneven surfaces. Yet for another example, aircraft cabins are extremely tight and narrow. To fully automate the cleaning and/or disinfecting a place by using cleaning and/or disinfection robots, the robots have to be flexible enough to navigate through different environment.

Yet, existing cleaning and/or disinfection robots are usually heavy or bulky, and cannot be deployed at sites that have limited space or without the assistance of lifting machine or substantial manpower.

Further, existing disinfection robots that apply disinfectants by way of spraying or wet fogging may take 4-6 hours to complete the disinfection process and allow the disinfection droplets to settle or exhaust to a safe level before the area may be re-opened for entry. In respect of mass transit, its available time for cleaning and disinfection could be very short if the mass transit is subject to a tight schedule having limited time between rides. This is particularly true for aircrafts which have to depart soon after their arrival at the airport and be ready for the next flight.

In light of the foregoing background, it is desirable to have a mobile robot that may be conveniently deployed for cleaning, disinfecting and sterilizing various places, including public space in mass transits such as aircraft cabin and railway system, and to reduce the conventional turnaround time for disinfection to meet the demand. It is also desirable to have disinfection methods that effectively and efficiently disinfect confined spaces to combat the infection.

### SUMMARY

Described herein are devices, systems and methods for cleaning and/or disinfection of spaces by robotic means.

It is an aspect of the present invention to provide a cleaning and disinfection mobile robotic system to enhance consistency and reliability when it comes to cleaning and disinfection.

It is yet another aspect of the present invention to reduce the time and cost of cleaning and disinfection, helping to bring back public confidence in public transits. Frequent cleaning and disinfecting areas in connection with public transits is an important element to re-establish public confidence in public transits.

Prior to the present invention, there is no automated disinfection robot that is narrow and small enough to navigate through the narrow train and aircraft cabins, yet carries heavy payload and performs disinfection effectively and efficiently. The present invention provides robots that may be conveniently deployed at public transits and navigate through small and narrow spaces such as train and aircraft cabins, and performs cleaning and disinfection effectively and efficiently.

It is an aspect of the present invention to provide small foot-print, precisely controlled and reliable robots for effective and efficient disinfection. The robots of the present invention are light and small, and are able to navigate through small and narrow spaces. In some embodiments, the width of the robot of the present invention is ranged from about 32 centimeters (cm) to about 34 cm. In some embodiments, the weight of the robots of the present invention is in the range of 10 kilograms (kg) to 15 kg without disinfectant and battery.

It is an aspect of the present invention to provide robots that apply disinfectants to the surrounding with high coverage and adjustable spray rates. It is yet another aspect of the present invention to enhance the effectiveness and efficiency of disinfection of spaces such that the spaces may be sufficiently disinfected yet quickly and safely reopened for entry.

It is an aspect of the present invention to provide an atomization system that is capable of generating fine droplets (i.e. liquid of small particle size) for spraying or fogging including cold fogging and thermal fogging. In some embodiments, the present atomization system creates atomization from a variety of mechanical means, which includes but is not limited to ultrasonic and electrostatic processes. In one embodiment, the present atomization system generates fine droplets of particle size ranging from about 2 microns to about 40 microns, and preferably about 2 microns to about 10 microns. In yet another embodiment, the droplets generated by the present atomization system are substantially uniform in size, with at least 95% of the droplet population having substantially the same particle size.

Accordingly, embodiments of the present invention, in one aspect, may be a cleaning and disinfection robot that may be conveniently deployed at public transits and performs cleaning and disinfection quickly and thoroughly, permitting the public transits be disinfected and safe to be reopened and stayed in shortly after the completion of disinfection process. In yet another aspect, the present invention provides a robotic system for cleaning and disinfection of spaces and a method of operating thereof.

It is another aspect of the present invention to provide methods for disinfecting a confined space such as a space on a railway system or an aircraft or a confined room. By providing a multiplicity of droplets of a disinfection composition to the confined space, and allowing the air and/or surfaces within the space to contact with an effective concentration of the disinfection composition for an effective period of time, the level of microorganisms in the air and/or on the surfaces within the space could be satisfactorily reduced.

In summary, the robots, systems and methods according to various embodiments of the present invention improve disinfection of spaces generally, and particularly allow disinfection of public transits in a convenient, effective, efficient and safe manner. The public, passengers and workers will all be benefited as a result.

Additional aspects of the invention will be set forth in part in the description which follows and, in part, will be obvious from the description, or may be learned by practice of the invention.

### BRIEF DESCRIPTION OF DRAWINGS

Persons of ordinary skill in the art may appreciate that elements in the figures are illustrated for simplicity and clarity so not all connections and options have been shown. For example, common but well-understood elements that are useful or necessary in a commercially feasible embodiment may often not be depicted in order to facilitate a less obstructed view of these various embodiments of the present disclosure. It may be further appreciated that certain actions and/or steps may be described or depicted in a particular order of occurrence while those skilled in the art may understand that such specificity with respect to sequence is not actually required. It may also be understood that the terms and expressions used herein may be defined with respect to their corresponding respective areas of inquiry and study except where specific meanings have otherwise been set forth herein.

FIG. 1 is a schematic view of a disinfection mobile robot comprising certain modules according to one embodiment.

FIG. 2 is a schematic view of a disinfection mobile robot according to another embodiment.

FIG. 3 is a schematic view of a disinfection mobile robot with locking means according to one embodiment.

FIG. 4 is a schematic view of a disinfection mobile robot comprising various modules according to one embodiment.

FIG. 5 is a schematic view of an atomization system of a disinfection mobile robot according to one embodiment.

FIG. 6 is a schematic diagram of a metro where 11 cross-sections (cross-section 1 to 11) were treated and monitored during the disinfection process.

FIGS. 7A to 7C are schematic diagrams of an aircraft cabin that is disinfected by the present invention according to one embodiment. FIG. 8 is a floor plan of an enclosed room of about 200 square feet and 8 feet in height, and is disinfected by a stationary disinfection robot according to one embodiment.

FIG. 9 is a floor plan of an enclosed room of about 320 square feet and 9 feet in height, and is disinfected by a stationary disinfection robot according to one embodiment.

FIG. 10 is a floor plan of an enclosed room of about 320 square feet and 498 cm in height, and is disinfected by an automated disinfection robot according to one embodiment.

FIG. 11 is a cross-section of a compartment with an electrostatic atomizer according to one embodiment.

FIG. 12 is a diagram illustrating 11 locations on a train selected for surface cleanliness test evaluating disinfection according to one embodiment of the invention.

FIG. 13 is a diagram illustrating locations on an airplane cabin for surface cleanliness test evaluating disinfection according one embodiment of the invention.

FIG. 14 is a diagram illustrating a floor plan of an enclosed room of about 330 square feet and 300 cm in height, and being disinfected by an automated disinfection robot according to one embodiment.

### DETAILED DESCRIPTION

Embodiments may now be described more fully with reference to the accompanying drawings, which form a part hereof, and which show, by way of illustration, specific exemplary embodiments which may be practiced. These illustrations and exemplary embodiments may be presented with the understanding that the present disclosure is an exemplification of the principles of one or more embodiments and may not be intended to limit any one of the embodiments illustrated. Embodiments may be embodied in many different forms and should not be construed as limited to the embodiments set forth herein; rather, these embodiments are provided so that this disclosure may be thorough and complete, and may fully convey the scope of embodiments to those skilled in the art. Among other things, the present invention may be embodied as methods, systems, computer readable media, apparatuses, or devices. Accordingly, the present invention may take the form of an entirely hardware embodiment, an entirely software embodiment, or an embodiment combining software and hardware aspects. The following detailed description may, therefore, not to be taken in a limiting sense.

Described herein are devices, systems and methods for cleaning and/or disinfection of spaces by robotic means.

It is an aspect of the present invention to provide a cleaning and disinfection mobile robotic system to enhance consistency and reliability when it comes to cleaning and disinfection, and to reduce the time and cost of cleaning and disinfection.

It is yet another aspect of the present invention to provide mobile robots that may be conveniently deployed at public transits and navigate through small and narrow space such as train and aircraft cabins, and performs cleaning and disinfection effectively and efficiently.

In yet another aspect, the present invention provides an atomization system that is capable of generating fine droplets (i.e. liquid of small particle size) for spraying or fogging.

In another aspect, the present invention provides a robotic system for cleaning and disinfection of spaces and a method of operating thereof.

In one aspect, the present invention provides methods for disinfecting a confined space using droplets of a disinfection composition. In another aspect, the present invention provides methods for reducing the level of a target microorganism in a confined space using droplets of a disinfection composition.

### Disinfection Robots

The present invention provides small foot-print, precisely controlled and reliable robots for effective and efficient disinfection. The robots of the present invention are light and small, and are able to navigate through small and narrow space.

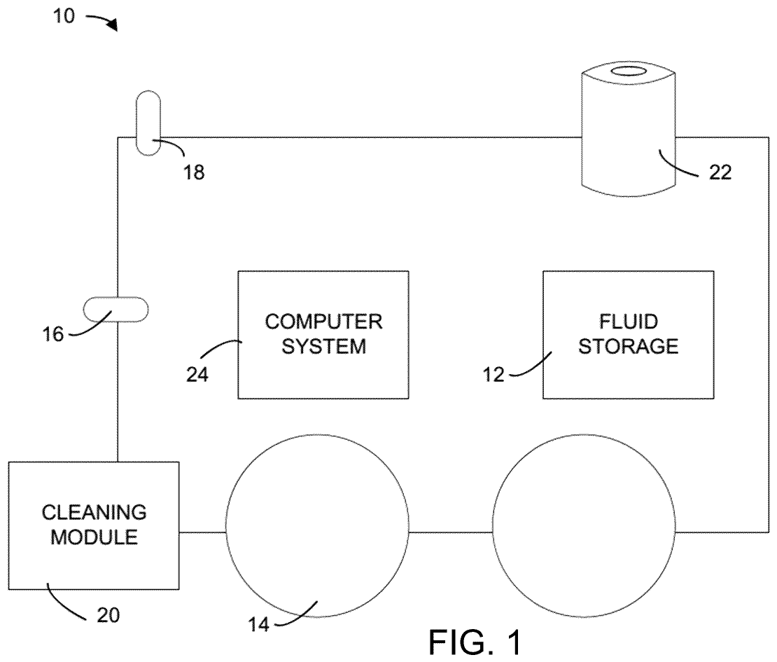

Referring to FIG. 1, the first embodiment of an automated cleaning and disinfection robot 10 comprises at least one fluid storage 12 configured to hold Quaternary Ammonium Compounds (QACs) and/or any other suitable disinfectant (“Disinfectant”), at least one wheel 14, at least one obstacle sensor 16 installed at the front or the side of the robot 10 configured to detect any obstacle in its path as it travels, at least one disinfectant sensor 18 configured to obtain the concentration of dispensed Disinfectant in an area and/or volume, a cleaning module 20 configured to remove visible foreign matters from a surface, a disinfection module 22 configured to apply Disinfectant to kill airborne germs and/or germs on the surface and a computer system 24 configured to receive the signals from the sensors 16, 18, control the disinfection module 22 based on the signal received from the disinfectant sensor 18, and navigate through a predetermined area while avoiding obstacle based on the signal from the obstacle sensor 16.

There are many factors affecting the biocidal efficacy, for example, the materials to be disinfected, the temperature, the humidity and the pressure. The computer system 24 of the present invention may use machine learning to optimize the biocidal efficacy and the amount of Disinfectant used based on the surrounding factors thereby avoiding over-use of Disinfectant. Optimization of Disinfectant may reduce the pollution and the associated risk to the public.

In some embodiments, an adherence chemical (e.g. polymer) may be added into the Disinfectant to allow it to stay on the surface longer.

In some embodiments, the robot 10 may include any rechargeable battery. In yet some embodiments, the robot 10 includes lithium battery.

In some embodiments, the robot 10 may include at least one motor configured to drive the wheel 14.

In some embodiments, the obstacle sensor 16 may be a medium range scanner and may give the robot 10 a 360-degree visibility for at least 5 meters. In some embodiments, the scanner may be light sourced (e.g. laser) or an audio-based (e.g. sonar). In some embodiments, the obstacle sensor 16 may be a camera and/or sonar transducer.

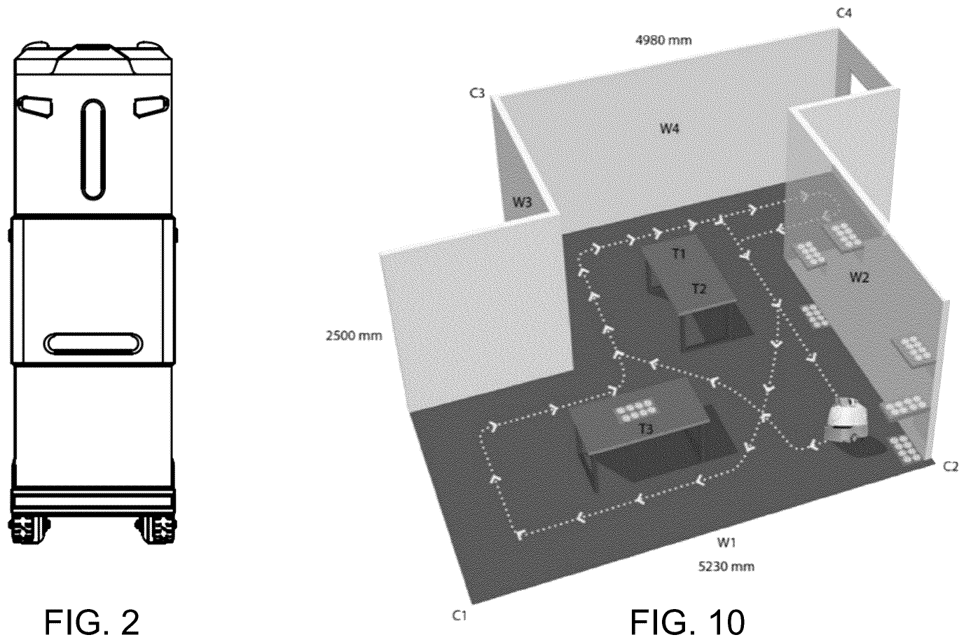

Referring to FIG. 2, the robot of the present invention has an elongated body enabling it to be deployed at and navigate through narrow spaces such as aisles of aircraft cabins. In some embodiments, the width of the present robot is no more than 34 cm, and preferably ranged from about 32 cm to about 34 cm.

In some embodiments, the weight of the present robot is less than 20 kg without disinfectant and battery and preferably less than 10 kg without disinfectant and battery. In one embodiment, the weight of the robot without disinfectant and battery is in the range of about 10 kg to about 15 kg.

In some embodiments, the present robot comprises at least two wheels, where one of the two wheels is disposed on one side of the chassis and the remaining wheel is disposed on the opposite side of the chassis. In some other embodiments, the present robot comprises at least four wheels, where two of the wheels are disposed on one side of the chassis and the remaining two wheels are disposed on the opposite side of the chassis. In some embodiments, the present robot further comprises one or more motors (or equivalent electronic components) configured to drive its one or more wheels.

In some embodiments, the present robot is able to move around narrow paths, turn at the spot in 360-degree (i.e. skid steering) and/or steer around tight corners (90 degrees). In some other embodiments, the robot is capable of climbing to 15 degree ramp.

In yet another aspect of the present invention, the present invention connects and combines two independent 2 wheel-drive systems to form a 4 wheel-drive system, where the wheels of the two independent 2 wheel-drive systems may be of identical or different diameters and configuration.

In some embodiments, the chassis of the present robot is made of at least one metallic material, at least one plastic material or a combination thereof. Examples of metallic materials include metals and alloys, including but not limiting to aluminum, copper, brass, stainless steel, titanium and steel. Examples of plastic materials include but are not limited to plastic materials falling within rating V0 of the UL94 Flame Classification (which is the standard for Safety of Flammability of Plastic Materials for Parts in Devices and Appliances testing), and other plastic materials that are in compliance with the industry standard.

The navigation or movement of the robot of the present invention may be operated manually (e.g. through a handle) or under a navigation system (e.g. through motors and computer system).

In some embodiments, the present robot further comprises a handle through which the user may push, pull or drag the robot by hand. In one embodiment, the handle is a foldable telescope handle. In one embodiment, after the user has moved the robot to a desirable position in a space, the user may leave the space and the robot will carry out the disinfection process automatically.

In some embodiments, the present robot is an autonomous disinfection mobile robot where the robot navigates under an automated navigation system and performs disinfection automatically.

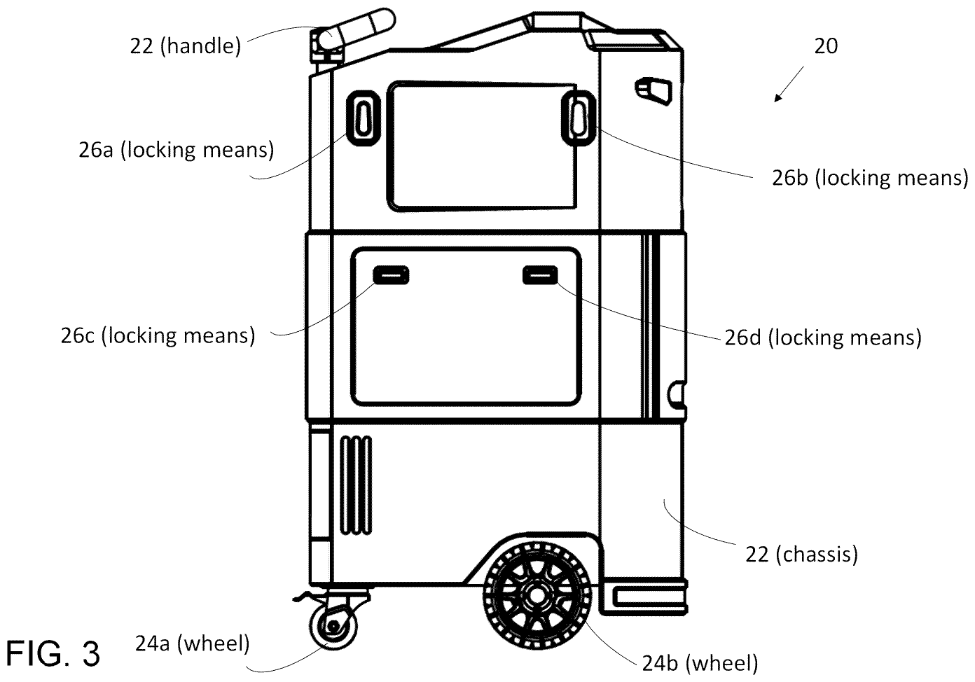

Referring to FIG. 3, the present robot 20 may further comprise a handle (22) and one or more locking means (26a, 26b, 26c and 26d) configured to pair with one or more complementary components on a strap or straps, enabling the users to carry the robot on their shoulders. In one embodiment, the handle 22 may enable the robot 20 to be operated in a manual mode. The robot 20 may also provide a display (not shown) on top of an exterior surface of the robot 20 to display a variety of status or information of the robot 20. For example, the status or information may include battery level, disinfection level, disinfection plan(s), or the like. In another embodiment, the robot 20 may include one or more modules that may be side panels or doors 28 to house various modules to further enhance the capabilities of the robot 20.

The locking means and its complementary component may be in the form of a hook and a loop, or a side release buckle. In one embodiment, a user may connect the locking means of the present robot and the corresponding complementary components on the strap(s), and then carrying the robot on their body via the strap(s). In yet another embodiment, other articles such as a notice or display board may be attached to the present robot using a similar setting of the locking means and complementary components described herein.

In some embodiments, the robot 20 may further include a set of wheels 24a and 24b to enable the robot 20 to move or be moved from locations to locations. In one embodiment, the wheels 24a may be in a smaller size than the wheels 24b. In one embodiment, the wheels 24a may move about in 360 degrees to facilitate navigation or maneuvering of the robot 20. In another embodiment, the robot 20 may further include a chassis 22 to assist the maneuvering of the robot 20. For example, the chassis 22 may include motorized or powered device to provide a semi-power-assisted drive when the robot 20 is energized or is in operation.

In some embodiments, the powering module comprises a power plug for obtaining electricity from an external power source and at least one battery configured to store energy enabling the robot to operate without connecting to any external power source. In one embodiment, the battery is a lithium battery, alkaline battery and the like.

In some embodiments, the battery is detachable form the robot body and is changeable and/or rechargeable. With the changeable battery setting described herein, users may quickly and easily change the battery, enabling the present robot to operate constantly almost without interruption and without the need to wait for recharging.

In some embodiments, the battery may be recharged when it is docked on the present robot, and the recharging may be done manually (e.g. by providing external power through the power cord) or automatically (e.g. by directing the robot to obtain external power from an external charging dock). In some other embodiments, the present robot or the battery is capable of being recharged via an external charging dock through wired or wireless means. In yet another embodiment, the battery may be recharged when it is detached from the present robot.

In some embodiments, the battery may run for at least 2 hours after it is fully charged. According to some embodiments of the present invention, the disinfection process where the dispersion of disinfection droplets may take 30 minutes to 60 minutes to complete, and the present robot may be deployed to perform at least two rounds of disinfection without recharging or changing the battery.

### Disinfection Module

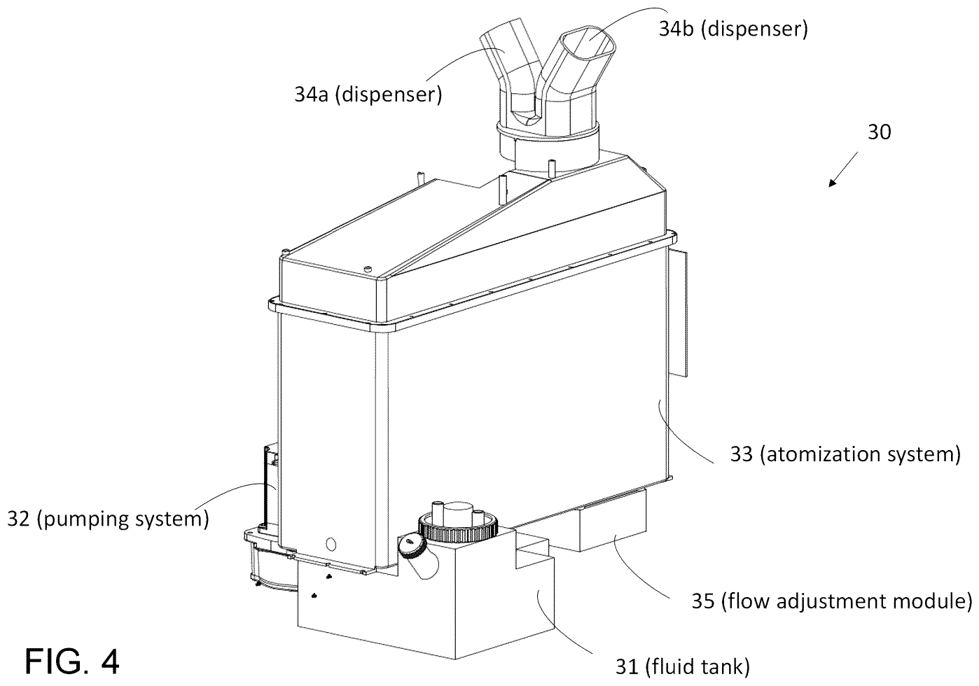

Referring to FIG. 4, the disinfection module (30), which may be housed in the robot 20, may include at least a fluid tank (31) configured to store a disinfectant liquid, a pumping system (32) configured to drive the movement of the disinfectant liquid within the robot 20, an atomization system (33) configured to generate droplets of disinfectant, an dispenser (34) configured to dispense or apply the droplets of disinfectant from the robot 20 to its surrounding and a flow adjustment or actuator module (35) configured to control the flow rate and direction of the disinfectant droplets. In yet another embodiment, the disinfection module may comprise two or more fluid tanks for holding the same or different type of fluid or disinfectant. In another embodiment, the configuration or housing of the disinfection module 30 may be elongated or be slender in shape to fit into the robot 20. In one example, the robot 20 may be designed to be navigated along isles between seats in a confined space, such as trains, airplanes, or the like. Therefore, the dispenser 34a and 34b may be positioned in a position higher or on top of the disinfection module 30. In this embodiment, for example, the dispensers 34a and 34b outlets may be positioned on top of the atomization system 33. In a further embodiment, the dispensers 34a and 34b may be in opposite directions to increase the exposure of the disinfectant.

In one embodiment, the fluid tank may store at least 5 liters of liquid. In some embodiments, the fluid tank may store about 3 liters to about 5 liters of liquid. According to some embodiments of the present invention, about 5 liters of disinfectant will be utilized in a disinfection process of 60 minutes, a fluid tank of 5 liters will therefore enable the present robot to supply disinfectant continually for up to 60 minutes.

In one embodiment, the pumping system comprises at least one pump which is electrically driven and configured to drive the flow of the liquid within the robot.

### Atomization System

In one aspect, the present invention provides atomization systems that may generate fine droplets of disinfectant for spraying or fogging application. In various embodiments, the droplets of disinfectants can be applied at various temperatures depending on the need, and could be by way of cold fogging or thermal fogging.

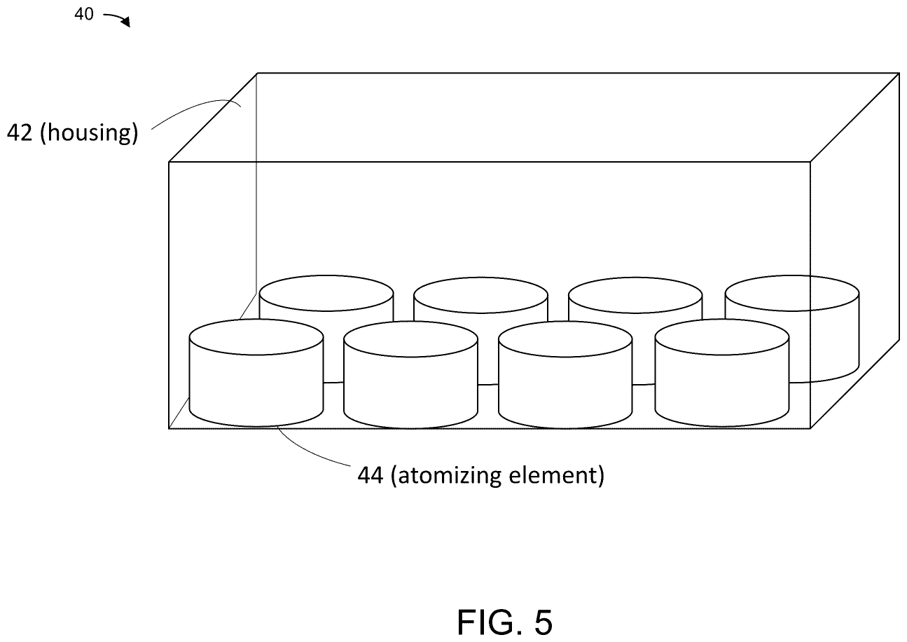

Referring to FIG. 5, the atomization system (40) may include at least a housing (42) for holding at least one disinfectant liquid, one or more atomizing elements (44) configured to convert liquid into fine droplets and at least one outlet allowing the droplets to travel from the housing to the dispenser. In one embodiment, the atomization system 40 may be positioned within the disinfection module 30. In another embodiment, the atomization system 40 may be positioned outside the disinfection module 30 but still housed within the robot 30 within the robot's compartments (accessible via the side panels 28). In one embodiment, the housing comprises one or more chambers for holding one or more type of disinfectants.

In some embodiments, the atomizing elements 44 may be an ultrasonic transducer.

In one embodiment, the atomizing element comprises an atomizing surface and a generator configured to cause vibration by converting high frequency sound waves into mechanical energy that is transferred into a liquid, such that the liquid will be broken into fine droplets when it contacts the atomizing surface.

In yet another embodiment, the atomizing element comprises a probe where the liquid may flow through and a generator configured at the tip of the probe to cause vibration using high frequency sound waves thereby converting the liquid reaching the tip into fine droplets

In one embodiment, the high frequency sound wave is an ultrasound.

In some embodiments, the present atomizing element being an ultrasonic transducer is configured with a special echo structure and hole size and is capable of generating droplets that are substantially uniform in size.

In some other embodiments, the atomizing element is an electrostatic atomizer 1102 that comprises a discharge electrode and a voltage generation unit configured to generate a pulse voltage to be applied to the discharge electrode, where the liquid will be atomized into fine droplets when it is in contact with the discharge electrode. In one embodiment, the atomizing element comprises a surface that is coated with a plurality of negative ions or charged particles, thereby inducing the liquid into fine droplets.

In one embodiment, the droplets generated by the present invention bear positive or negative charges to enhance their adhesion to surfaces. In some embodiments, the present invention further comprises at least one electrode, one ionizer or similar component that is configured to make the disinfectant droplets charged, where such electrode, ionizer or component may be located within or outside of the atomizing element. In one embodiment, the disinfectant droplets are made charged when the disinfectant solution is converted into droplets by the present atomizing element. In yet another embodiment, the disinfectant droplets are made charged after they are generated using the present atomizing element. For example, an ionizer may be placed in the dispenser to cause the droplets travelling along the dispenser to become charged.

Existing atomizing technologies including typical ultrasonic atomizers cannot produce sufficiently uniform mist or fog, with size of the droplets generated widely spreading from 5 microns to 15 microns. The atomizing system is capable of generating very fine droplets of sufficiently uniform size. In one embodiment, the atomizing system 40 described herein may generate droplets of size ranged from about 2 microns to about 10 microns. In yet another embodiment, the present atomizing system may generate droplets of size from about 20 microns to about 40 microns.

In one embodiment, the atomizing system 40 may generate droplets with at least 95% of the droplet population having substantially the same particle size.

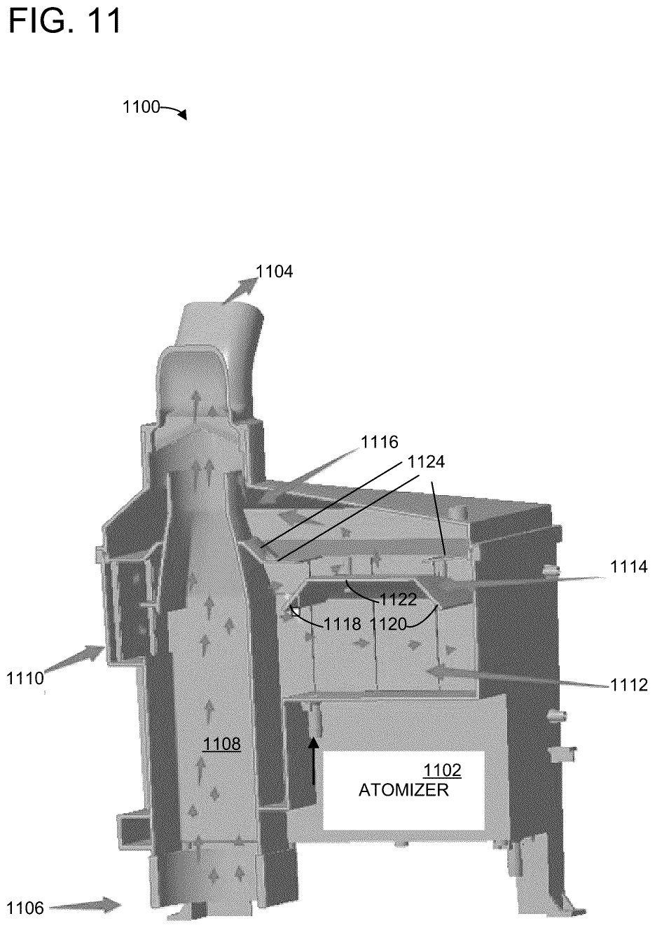

Referring to FIG. 11, a configuration system 1100 may be employed along with the atomizing system 40. The system 1100 may include a system of channels or paths for the optimal sized droplets may exit an outlet 1104 (e.g., dispensers 34a and 34b). In one embodiment, an air propelling source (e.g., a fan) may provide a source of forced air through a first channel 1108. The arrows in the channel 1108 may show how air flows through the channel 1108. A side channel 1110 may direct the air from the channel 1108 to another chamber 1112. In one embodiment, the droplets from the atomizer 1102 may first enter the chamber 1112. With the air flow direction as shown by the arrows in the chamber 1112, the droplets may ride with the air to flow from the chamber 1112 toward a choke point or a throttle 1116. In one example, the air flow direction may be horizontal or generally horizontal in nature to direct the droplets to pass a blocker or a blocking plate 1114. In one aspect, the blocking plate 1114 may prevent, block, or restrict larger sized droplets from escaping the chamber 1112.

In one aspect, the blocking plate 1114 may include a first plate 1122 and side plates 1118 and 1120. The side plates 1118 and 1120 may be angled with respect to the first plate 1122. For example, as shown in FIG. 11, the side plates 1118 and 1120 may be angled toward the atomizer 1102 and away from the outlet 1104. In another embodiment, the side plates 1118 and 1120 may be angled away from the atomizer 1102 and toward the outlet 1104. In one embodiment, the system 1100 may further include one or more plates 1124. In one example, the one or more plates 1124 may be positioned in various areas to further restrict those droplets that escaped the blocking plate 1114. For example, as shown in FIG. 11, the one or more plates 1124 may be disposed approximate to the blocking plate 1114. In the example as shown, the one or more plates 1124 may be disposed above the blocking plate 1114 and may be generally horizontal.

In one aspect, as the smaller size or very small size droplets travel toward the outlet 1104, the choke point or throttle 1116 may further restrict or reduce the size of the droplets toward the outlet 1116. By the time the droplets reach the channel 1108, the force of the air may carry the droplets to exit the outlet 1104.

Due to the very small size of the droplets produced by the atomizing system 1100 described herein, the present invention is capable of dispensing disinfectant by way of dry fogging which has certain benefits over the approach of spraying or wet fogging. First, dry fogging achieves high air coverage and is suitable for both air and surface disinfection. Second, the amount of disinfectants to be used may be significantly reduced and may be up to 80% disinfectants according to some embodiments of the present invention, thereby reducing potential pollution and the potential associated risk to the public.

Further, dry fogging will not cause the objects in contact with the droplets wet, therefore in the case of disinfection of aircraft cabins or train cabins where the seat covers are typically made of cotton or other materials that tend to absorb moisture, it will not require a long time or extra step to let the treated objects or surfaces dry, thereby enabling the public transits to be used shortly after the disinfection.

Further, the present atomizing system requires less power to operate and therefore reduces the power requirement of the entire device.

### Dispenser

In one embodiment, the present robot comprises a dispenser configured to dispend the disinfectant droplets from the robot to its surrounding. In one embodiment, the dispenser comprises at least one outlet connected to the exterior of the chassis at one end and connected to the atomizing system at the other end. In one embodiment, the dispenser comprises two outlets as depicted in FIG. 4, where the two outlets are configured to spray the droplets at different angles in the range of zero degree to 90 degrees as measured from horizontal level. The end of the outlet connecting to the exterior may be of various shape and dimensions, depending on the coverage and rate of spraying to be achieved.

In one embodiment, the dispenser is detachable from the robot, allowing the users to switch between dispensers of various configuration.

The dispenser may be made of plastic materials, including but not limited to plastic materials falling within rating V0 of the UL94 Flame Classification

### Flow Adjustment Module

As depicted in FIG. 4, the present robot further comprises a flow adjustment module configured to control the flow rate and/or flow direction of the disinfectant droplets. In some embodiments, the present flow adjustment module comprises at least one fan with fixed speed or adjustable speed. As such, the flow rate of the droplets within the robot and also the spraying rate of the disinfectant droplets may be adjusted according to the users' instructions.

Depending on the need and other factors such as the size and design of the space, the spraying rate of the disinfectant in one disinfection process may be constant or varying. In some embodiments, the spraying rate of the disinfectant is in the range of 2,000 mL/min to 8,000 mL/min, and preferably 4,000 mL/min to 5,000 mL/min.

Depending on the need and other factors such as the size and design of the space, the droplets may be dispensed to the surrounding with or without the operation of the fan. In some embodiments, the fan is in operation during the course of dispersion. In some other embodiments, the fan is turned off during the course of dispersion and then turned on immediately or shortly after the dispersion to direct the flow and dispersion of the droplets within the space.

With the special design of the dispenser and/or flow adjustment module described herein, the flow and dispersion of droplets may be precisely controlled to ensure effective disinfection of the space.

The present robot is capable of dispensing droplets reaching the ceiling and walls of the indoor space for effective disinfection.

In particular, the present robot may achieve a high coverage that is sufficiently wide for disinfecting in space within public transits. In some embodiments, the present robot is capable of covering a cross-sectional area of at least 3 meters×2.5 meters (3 m×2.5 m). In some embodiments, the droplets produced by the present robot may reach up to 2.5 m above the upper end of the robot, and up to 1.5 m to the left from the center of the robot and up to 1.5 m to the right from the center of the robot. These droplets may then reach to further location within the space by way of diffusion.

The robot described herein is compatible with various type of aqueous-based disinfectants and suitable for disinfecting a wide range of objects. Disinfectants that may be used in couple with the present robot includes but are not limiting to hypochlorous acid, chlorine dioxide, sodium hypochlorite, potassium hypochlorite, sodium dichloroisocyanurate, potassium dichloroisocyanurate, hydrogen peroxide, formaldehyde based disinfectant and quaternary ammonium chlorides.

The present robot may further comprise one or more additional disinfection modules that employ other mode or technologies of disinfection. Examples of such additional mode or technologies of disinfection may include ozone-free ultraviolet-C (UVC) disinfection and air purifier.

The present robot may further comprise a cleaning module configured to remove visible foreign matters such as dirt and trash from the surfaces to be cleaned. In some embodiments, cleaning may be carried out using the present robot with a cleaning module or manually prior to the disinfection process.

In some embodiments, the present robot may operate with a low noise level at 50 dB or less.

### Sensors and Autonomous Navigation System

The present robot may further comprise a navigation module configured to guide the navigation of the robot and enable the robot to operate and navigate in narrow spaces such as aisles of aircrafts under an autonomous system.

Autonomous or automated robots are usually installed with sensors to receive signals about the surrounding environment and some controller system to adjust the movement or operation of the robot based on the received signals such that the robot may navigate along a predetermined route while avoiding obstacles. Usually, a tolerance distance is set to restrict the robot from further navigating or to direct the robot to adjust its route when the sensor(s) detect the presence of obstacles within the pre-determined tolerance distance. While setting a longer tolerance distance will likely reduce the chance of hitting obstacles during the navigation, a too long tolerance distance could significantly hinder the performance of the robot as the robot has to stop or adjust its pre-determined route even when the chance of hitting the obstacles is remote. When it comes to navigation within a narrow space and/or limited light such as in an aircraft cabin, high maneuverability and precision are great challenges. With the special design described herein, the present robot is capable of navigating within narrow spaces with high precision in the sense that it follows the pre-determined route with little derivation and with a high tolerance to its neighboring obstacles.

In one embodiment, the present robot further comprises at least one sensor which receives signals from the surrounding environment for the purposes of real-time monitoring and/or positioning. Sensors to be used in couple with the present robots may be light sourced (e.g. laser system, LiDar system and camera), audio based (e.g. sonar system), radio-wave based (e.g. radar) and any other sensors that are applicable for automatic localization and navigation system. Signals and images collected or received from the sensors may be transmitted to any applicable signal processer for analysis and/or recorded as part of the activity logs.

In some embodiments, the one or more sensors described herein may give the present robot a 360-degree visibility from 5 meters to 40 meters and may detect obstacles located up to 40 meters from the robot.

In some embodiments, the present robot is configured to have a tolerance distance of 20 millimeters (mm) on the left and 20 mm on the right, meaning that the robot will continue to navigate and operate so long as there is no obstacle detected within 20 mm on both the left side and the right side.

Together with the wheels described herein, the present robot is capable of navigating and turning within narrow spaces autonomously and steadily with low chances of hitting its neighboring obstacles. Accordingly, the present robot will be able to follow its pre-determined route with little deviation.

### Safety Control

The present invention may further comprise one or more safety measures to ensure disinfection will be carried out safely, thereby reducing the potential health risks imposed to people.

In some embodiments, the present robot further comprises an identification module configured to identify and track disinfectants and/or operators. The identification module may comprise one or more scanners configured to detect or read certain signals from an identification tag and transmit such signals to one or more processors via electronic communication. The robot may request users to provide the identification tag and will only operate in full when the tag matches the approved list of disinfectants and/or operators stored in the system.

Examples of such identification tag include but are not limited to RFID, QR code, NFC chip and bar code. For example, individual RFID tag is assigned to each bottle of disinfectant solution that has been approved for use for disinfection by way of spraying or fogging (as the case may be) and the robot door will only be opened when the compatible RFID tag is presented. If a compatible RFID tag is not presented, the users will not be able to activate the robot or open the robot for filling in the disinfectant solution and performing the disinfection. Each RFID tag may allow one or more times of activation depending on the fluid tank size of the robot and the size of bottle carrying the disinfectant solution (e.g. multiple activation may be made possible if the bottle of disinfection solution cannot be finished in one time). In some other embodiments, the volume of liquid in the fluid tank are tracked on a real-time basis to ascertain the consumption of the disinfectant solution and such information may be used to update the records of the corresponding RFID tag. This will ensure that only the approved disinfectant solution will be used and/or the robots will be operated by the authorized persons.

In yet another embodiment, the present robot may further comprise an emergency safety control where the robot will stop moving or spraying immediately or within 1 second when the sensor detects moving objects in a pre-determined distance. In one embodiment, the robot will then deliver an alert signal to notify the users about the situation. This will reduce the risks of exposure of human or living organisms in case they are not removed from the space prior to the disinfection process.

### Operating System

In some embodiments, the present robot may be further connected to a communication network through a communication module. The communication network may further connect to a remote computer system where users may instruct the robot from a remote location through a computer, smart device or similar external devices. In some embodiments, the robot may share its cleaning and disinfection parameters, including but not limited to the data received by its sensors, with the communication network. In yet some other embodiments, the robot may receive instructions anytime during its operation via the communication network.

In some embodiments, the communication is a wireless communication including but not limiting to WI-FI, BLUETOOTH, NFC, and 4G or 5G telecommunication system.

It is an aspect of the present invention to provide a computer system configured to receive, process and/or analyze signals from the sensors of the present robot, to control the operation of the robot on a real time basis having regard to the real time data. In one embodiments, the computer system may use machine learning to identify the materials and/or type of objects (for example, the floor, the wall, the doors, seats, etc.) to be cleaned and/or disinfected based on the signals from the sensors to control and coordinate the operation of various modules of the robot, thereby adjusting and optimizing the performance of the robot. The computer system may further use such information to determine the optimized disinfection strategy based on previous data on cleaning and disinfecting similar objects.

In yet another embodiment, the computer system may further provide a monitoring system to record all activity logs for traceability. Information or activities to be recorded may include the start and end time of each operation, location and route of the robot, the operator in-charge, various events, incidents log, sprayed volume, and battery condition. In another embodiment, the computer system allows the users to create a cleaning and disinfection plan such that the robot will operate according to the users' pre-determine operational parameters such as the route, the spraying time, spraying rate, size of the droplets, and spraying mode.

The robot and computer system may be further configured to implement different modes of operation or functions. For example, the robot will be in a standby mode after is it powered up, or a setup mode allowing the user to communicate with the robot and give instructions via network communication using an external computer or smart device. In some other embodiments, there is provided an automated mode where the robot will navigate according to a pre-determined route, and a cleaning mode where the robot will perform cleaning using its cleaning module. In yet another embodiment, the robot may switch to an incident mode where it will stop operating and notify the users when it encounters problems such as shortage of power or solution, loss of connection, and presence of obstacles.

The computer system may include a microprocessor and a computer-readable storage medium or memory connected to the microprocessor.

Switches and buttons may also be provided on the robot for users to turn on/off the robot or control the robot. In the case of emergency, an emergency stop button to terminate any of the robot's operation may also be provided.

### Display and Alarm

The present robot may further comprise a display module configured to enable the users to receive information. Information to be displayed may include the start and end time of each operation, total time lapsed in minutes and seconds, GPS location of the robot, the operator in-charge, information about the disinfectant solution (e.g. the type of disinfectant solution being used, the volume being filled in the fluid tank, and the date and time of filling), and information about the fan and spraying (e.g. whether the fan is on and its speed, and the spraying rate), and the battery condition.

In some embodiments, the display module comprises an user interface allowing the users to access information at their own choice and give instructions pertaining to the operation of the robot.

In one embodiment, the display module is a LCD touch screen that is configured to show information about the operation of the robot such as the level of disinfection solution in the fluid tank, the battery level and spraying rate of the droplets on the one hand, and receive input from users on the other hand. In another embodiment, the display module may show icons about certain operational parameters where the users may pick on and control the operation of the robot accordingly.

In one embodiment, the present robot may further comprise one or more alarms configured to alert the users of any alarming situations that may affect the operation of the robot. For example, an audible alarm may be employed to notify the users in the event of low battery level, low solution level, overheating, detection of obstacles. Some other forms of alarms such as light indicators or announcement of messages may also be employed.

### Air Sampling Module and Detection Module for On-Site Monitoring

In one embodiment, the robot may further comprise an air sampling module or unit configured to collect air from the surrounding and a detection module configured to detect the presence or absence of one or more target substances or measure their level in the confined space. Such target substances include but, not limited to, microorganisms, volatile organic compounds (VOCs) and pollutants, and it is expected that the relevant information of such target substances may indicate the progress of the disinfection process and status of the confined space such as air quality therein.

In one embodiment, the detection module may be connected to the air sampling module. In another embodiment, the detection module may be integrated into the air sampling module.

The air sampling module may employ appropriate means for capturing the surrounding air such as a blower or pump to mechanically move air from the outside to the interior of the present robot, and at a fixed or variable speed and/or volume. In one embodiment, the air sampling module may comprise an inlet and a mechanism, such as a motor, for moving air from the surrounding into the body of the robot through the inlet, thereby collecting an air sample. The air sampling module may collect air samples continuously or at pre-determined intervals, during and/or after the operation of the present robot. In another embodiment, the detection module may employ any appropriate means for detecting the presence or absence of a target microorganism. For example, the detection module may further include a testing subsystem. For example, the subsystem may include a nucleic acid-based or antigen-based molecular testing device that may provide an indicia of the presence or absence of the target microorganism, and/or the quantity or similar indication of the target microorganism. In one embodiment, the detection module may include a signaling subsystem that may generate signals (which may be visual, electronic or other forms of signals) in view of the indicia from the testing subsystem. In one embodiment, the signaling subsystem may include a visual capturing device that captures images or indicia of the testing subsystem. In another embodiment, the signaling subsystem may be integrated with the testing subsystem so that the test subsystem may not provide the indicia to human. For example, the testing subsystem may transmit signals to the signaling subsystem before sending it to the detection module. In one embodiment, the detection module is capable of multiplex detection detecting two or more types of microorganism at the same time.

In another embodiment, the signaling subsystem may further connect with other subsystems, such as sensors that may capture raw data, such as temperature, humidity, location, or capture VOCs and pollutants.

In one embodiment, the detection module or the robot may further include a processor or a microprocessor coupled with a storage unit to perform analysis of the detected signal. For example, the processor may be capable of quantifying or semi-quantifying the target microorganism based on the detected signal, thereby notifies users the level of the target microorganism in the air in absolute or relative measurement units. In yet another embodiment, the present device may give alert signals through the signaling subsystem to notify users of any alarming situations.

The present detection module may carry out the detection continuously or at pre-determined intervals, during and/or after the operation of the present robot, and send the resulting data to the operating system of the robot. In one embodiment, the operating system of the present robot is configured to receive data or signals from the detection module and is capable of adjusting one or more operating parameters of the present robot such as the spraying rate and the spraying time in view of the data or signals received.

In some embodiments, some or all of the modules described herein are separable and detachable from the robot body, thereby giving flexibility to design and assemble the robot depending on the need.

### Disinfection of Aircraft Cabin Using Disinfection Robots

In various embodiments, the present invention provides a disinfection robot that is adapted for disinfection of aircrafts. The robots described here have a light and elongated body that enable them to be easily deployed at the narrow aisle of aircrafts. With the sensors and navigation system according to some of the embodiments, the robot may navigate along the narrow aisle with high precision and complete the disinfection process following the pre-determined route with little deviation. Further, the present robot is capable of disinfecting the surfaces and/air by way of dry fogging, thereby reducing the time required for the flights to return to its normal stage and be ready and safe for the next flight. The present robot does not rely on WI-FI connection to operate and is able to perform disinfection in the cabin according to the pre-set program.

Users may use the present robot to disinfect the aircraft cabins under different scenario and adjust the type of disinfectant, the duration and other parameters of the disinfection processes according to their need. For example, a longer duration of disinfection and ventilation may be implemented if the aircraft is idle and is not subject to a next flight on the same day. Where the aircraft is subject to a tight schedule and may have 1 to 1.5 hours between two flights, a quick yet effective disinfection may be performed by the present robot. Accordingly to some embodiments of the present robot, disinfection time may be as short as about 15 minutes and the ventilation time may be around 15-30 minutes, this will greatly reduce the turnaround time and enable the aircrafts to start the next flight as scheduled. Yet in another embodiment, disinfection before meal using acceptable disinfectants and droplets of acceptable particle size may be employed. With frequent and adequate disinfection, the risks of infection or spreading the disease could be significantly reduced.

### Methods of Disinfection of Confined Spaces

It is an aspect of the present invention to provide methods for disinfecting spaces of various settings, including the air and surfaces within those spaces. In some embodiments, there are provided methods for disinfecting spaces in a confined area such as the spaces on metro, trains, aircrafts, ships, and rooms such as hotel rooms, hostel rooms and rooms in workplaces or schools. In yet another aspect, there is provided a method of reducing the quantity, cell viability and/or infectivity of a target microorganism in a confined area or space.

In one aspect, the present invention provides a method of reducing the quantity, cell viability and/or infectivity of a target microorganism in a confined area or space, the method comprises the steps of a) dispersing into the area a multiplicity of droplets of a disinfectant composition, and b) allowing a time sufficient for the droplets to distribute throughout the area thereby maintaining an effective concentration of the disinfectant composition within the area for a period of time.

As used herein, cell viability of a target microorganism is a measure of the proportion of live, healthy cells of the target microorganism within a population.

As used herein, an effective concentration in respect of a disinfectant composition and a target space or surface refers to a concentration of the active ingredient in the disinfectant composition in the target space or on the target surface that can reduce the bioburden of the target microorganism by a satisfactory level. In some embodiments, the effective concentration of a disinfectant composition is a concentration of the active ingredient in the disinfectant composition in the target space or on the target surface that can reduce the bioburden of the target microorganism by 90-99%, or preferably by 95-99%. In some other embodiments, the effective concentration of a disinfectant composition is a concentration of the active ingredient in the disinfectant composition in the target space or on the target surface that can reduce the bioburden of the target microorganism by 99.1-99.99%, 99.96-99.99% or preferably 99.9-99.9%. In yet other embodiments, the effective concentration of a disinfectant composition is a concentration of the active ingredient in the disinfectant composition in the target space or on the target surface that can reduce the target microorganism by 1 log, 2 log, 3 log, 4 log, 5 log or 6 log within a specified period of time.

As used herein, the term “bioburden” refers to the number of viable microorganisms on a surface or in the air within a space prior to disinfection, and may reflect the degree of microbial contamination or load of that surface or space.

As used herein, the term “microorganism” refers to any non-cellular or unicellular (including colonial) organism. Microorganisms include all prokaryotes and can be pathogens. Microorganisms include bacteria, spores, lichens, fungi, protozoa, virinos, viroids, viruses, phages, and some algae. The microorganism may be a pathogenic microorganism or a non-pathogenic microorganism.

The time sufficient for the multiplicity of droplets of the disinfectant composition to disperse into an area and reach or maintain the effective concentration, may be a function of several factors, including but not limited to: the size of the droplets as they are dispersed, the type of disinfectant composition being used, the concentration of the disinfectant composition in the droplets, the identity of the target microorganism, the size and humidity of the area, and the pre-treatment conditions of the area. A calculation of the time may be made based on one or more of the above factors.

In some embodiments, the droplets of disinfectant composition are dispersed to the target space or surface by way of spraying or fogging, where droplets of disinfectant are dispersed in the air and land on the target surfaces, thereby killing or otherwise destroying microorganisms in the air and on such surfaces. Without being bound by any particular theory, fogging differs from spraying in terms of the size of the droplets which directly affects surface contact behaviour and spray distribution of the droplets. Typically, fogging disinfection devices generate droplet sizes between 10-40 microns and spraying devices generate larger droplets of size exceeding 100 microns. In some embodiments, the size of the droplets is in the range of about 2 microns to about 40 microns. In other embodiments, the size of the droplets is about 100 microns or above.

The spraying or fogging can be performed manually (e.g. by handheld misters) or by automated means (e.g. by hand free spraying/fogging devices, no matter stationary or mobile). In one embodiment, the droplets are dispersed using any of the robots, systems or inventions described herein.

The spraying or fogging may be applied at room temperature, a lowered temperature or an elevated temperature. In one embodiment, the droplets are dispersed by way of cold fogging or thermal fogging.

In some embodiments, the droplets are electrostatically charged.

In some embodiments, before the droplets of disinfectant composition are dispersed to the target surface, pre-cleaning of the surface is performed to remove organic matters and other obstructing objects.

Various types of disinfectant composition can be used in connection with the methods herein, provided that such disinfectant composition is applicable and safe for application by way of spraying or fogging. Examples of applicable disinfectant compositions include but are not limited to quats (quaternary ammonium compounds)-based disinfectants, hydrogen peroxide-based disinfectants, and chlorine-based disinfectants. In some embodiments, the disinfectant composition contains at least one active ingredient selected from the group consisting of quaternary ammonium compounds, hydrogen peroxide, sodium hypochlorite, hypochlorous acid, chlorine dioxide, potassium hypochlorite, sodium dichloroisocyanurate, and potassium dichloroisocyanurate.

The confined space to be disinfected by the present method can be of various size and settings. In one embodiment, the confined space has a size of about 500 cubic feet to 5,000 cubic feet. In yet another embodiment, the confined space has a size of about 1,500 cubic feet to 4,000 cubic feet.

In one embodiment, the confined space to be disinfected by the present method has all or some of its doors or windows closed or sealed. In another embodiment, air ventilation in the confined space is restricted; for example, one or more of the air-conditioning, vent or fan therein are turned off.

The present method of disinfection can be applied to a wide range of spaces. The following detailed description provides various embodiments to illustrate methods for disinfecting spaces on railway systems, aircrafts and confined rooms using the present invention.

### Disinfection on Railway System

In one aspect, there is provided a method of disinfecting a space on a railway system such as a metro, subway, train and the like. In yet another aspect, there is provided a method of reducing the quantity, cell viability and/or infectivity of a target microorganism in a space on a railway system. A space on a railway system may be a compartment of a metro, subway or train.

The method comprises the steps of a) dispersing into a space on a railway system a multiplicity of droplets of a disinfectant composition, and b) allowing a time sufficient for the droplets to distribute throughout the space thereby maintaining an effective concentration of the disinfectant composition within the space for a period of time. As described herein, the droplets can be dispersed manually or by automated means, and various types of disinfectant may be used.

### Numbered Embodiment

Embodiment 1. A method of reducing the quantity, cell viability and/or infectivity of a target microorganism in a confined space, comprising:
(a) dispersing into the confined space a multiplicity of droplets of a disinfectant composition; and
(b) allowing a time sufficient for the droplets to distribute throughout the confined space thereby maintaining an effective concentration of the disinfectant composition in the confined space for a period of time.

Embodiment 2. The method of embodiment 1, wherein the effective concentration is a concentration of the disinfectant composition in the confined space that is sufficient to reduce the bioburden of the target microorganism in the confined space by 90.0%-99.9% within the period of time.

Embodiment 3. The method of embodiment 1, wherein the effective concentration is a concentration of the disinfectant composition in the confined space that is sufficient to reduce the target microorganism in the confined space by 1 log to 6 log within the period of time.

Embodiment 4. The method of embodiment 1, wherein the effective concentration is a concentration of the disinfectant composition in the confined space that is sufficient to improve the surface cleanliness of the confined space thereby meeting an acceptable criteria of a relative light unit (RLU) as measured by a ATP bioluminescence assay.

Embodiment 5. The method of embodiment 1, wherein the confined space is an aircraft cabin, an enclosed space located on a rapid mass transit system, or an enclosed room.

Embodiment 6. The method of embodiment 1, wherein the size of the droplets is in the range of 2 microns to 40 microns.

Embodiment 7. The method of embodiment 1, wherein the disinfectant composition contains at least one active ingredient selected from the group consisting of quaternary ammonium compounds, hydrogen peroxide, sodium hypochlorite, hypochlorous acid, chlorine dioxide, potassium hypochlorite, sodium dichloroisocyanurate, and potassium dichloroisocyanurate.

Embodiment 8. The method of embodiment 1, wherein the effective concentration is in the range of 10 ppm to 40 ppm.

Embodiment 9. The method of embodiment 1, wherein the period of time is in the range of 15 seconds to 360 minutes.

Embodiment 10. The method of embodiment 1, where is the confined space is an aircraft cabin having the same or substantially the same configuration as the cabin of Boeing aircraft or an Airbus aircraft.

Embodiment 11. The method of embodiment 10, wherein the disinfectant composition is a hypochlorous acid-based disinfectant composition, wherein the effective concentration of the disinfectant composition is 10-40 ppm, and wherein the period of time is 15-30 minutes.

Embodiment 12. The method of embodiment 1, wherein the confined space is an enclosed room having a size of about 1,500 cubic feet to about 10,000 cubic feet.

Embodiment 13. The method of embodiment 1, wherein the confined space is an enclosed space located on a rapid mass transit system having the same or substantially the same configuration as the train compartment in model Metro Gammell EMU (DC) (M-train), Adtranz-CAF EMU, Rotem EMU, CNR Changchun EMU (C-train), SP1900/1950 EMU, Metro Gammell EMU (AC), Hyundai Rotem EMU (R-stock) or light rail rolling stock.

Embodiment 14. The method of embodiment 1, wherein the droplets are substantially uniform in size.

Embodiment 15. The method of embodiment 1, wherein the multiplicity of droplets is dispersed by way of spraying or fogging.

Embodiment 16. The method of embodiment 1, wherein the multiplicity of droplets is dispersed into the confined space continuously or non-continuously during the period of time.

Embodiment 17. The method of embodiment 1, wherein the target microorganism is selected from the group consisting of bacteria, spores, lichens, fungi, protozoa, virinos, viroids, viruses, phages, and algae.

Embodiment 18. The method of embodiment 1 wherein air ventilation in the confined space is restricted.

Embodiment 19. The method of embodiment 1, wherein the dispersing of the multiplicity of droplets is provided and controlled by an aerosol generating device located in the confined space.

Embodiment 20. The method of embodiment 1, further comprising a step of removing organic matters and obstructing objects in the confined space prior to the dispersing step.

Embodiment 21. The method of embodiment 1, further comprising the following steps after the commencement of the dispersing step:
(a) detecting the concentration of the active ingredient of the disinfectant composition in the confined space at regular intervals or continuously during the dispersing step;
(b) comparing the detected concentration with a predetermined target concentration of the active ingredient of the disinfectant composition; and
(c) ceasing the dispersing step once the detected concentration has achieved a value equal to or larger than the predetermined target concentration for not less than 5 minutes.

Embodiment 22. A disinfection robot comprising:
(a) a chassis;
(b) at least two wheels, wherein one of the wheels disposed on one side of the chassis and the other wheel disposed on the other side of the chassis;
(c) a disinfection module configured to dispense disinfectant to a surrounding area; and
(d) a powering module configured to supple energy for operating the robot; wherein the robot has an elongated body and is adapted to dispense disinfectant to a narrow space automatically.

Embodiment 23. The disinfection robot of embodiment 22, wherein the disinfection module comprises:
(a) a fluid tank configured to store at least one type of disinfectant liquid;
(b) a pumping system configured to drive the movement of the disinfectant liquid within the robot;
(c) an atomization system configured to generate droplets of disinfectant;
(d) a dispenser configured to dispense the droplets of disinfectant from the robot to its surrounding; and
(e) a flow adjustment module configured to control the flow rate and direction of the droplets of disinfectant.

Embodiment 24. The disinfection robot of embodiment 23, wherein the atomization system comprises one or more atomizing elements configured to convert the disinfectant liquid into fine droplets.

Embodiment 25. The disinfection robot of embodiment 23, wherein the atomization system generates the droplets of disinfectant by ultrasonic means, electrostatic means or a combination thereof.

Embodiment 26. The disinfection robot of embodiment 23, wherein the size of the droplets is in the range of 2 microns to 10 microns.

Embodiment 27. The disinfection robot of embodiment 23, wherein at least 95% of the population of the droplets generated has substantially the same size.

Embodiment 28. The disinfection robot of embodiment 22, wherein the robot is capable of generating droplets of disinfectant covering a cross-sectional area of at least 3 meters×2.5 meters.

Embodiment 29. The disinfection robot of embodiment 22, wherein the dispenser is detachable from the robot.

Embodiment 30. The disinfection robot of embodiment 22, wherein the rate of dispersion of the droplets of disinfectant is adjustable.

Embodiment 31. The disinfection robot of embodiment 22, wherein the robot further comprises an identification module configured to identify and track the disinfectant.

Embodiment 32. The disinfection robot of embodiment 22, wherein the robot further comprises a handle on one side of the chassis.

Embodiment 33. The disinfection robot of embodiment 22, wherein the powering module comprises at least one changeable battery.

Embodiment 34. The disinfection robot of embodiment 22, wherein the robot further comprises one or more locking means on one or more sides of the chassis, wherein the one or more locking means are configured to pair with one or more complementary components on one or more straps, wherein an user may carry the robot on their body via the one or more straps.

Embodiment 35. The disinfection robot of embodiment 22, wherein the weight of the robot is in the range of about 10 kg to about 15 kg.

Embodiment 36. The disinfection robot of embodiment 22, wherein the disinfection robot has a width of no more than 34 cm.

Embodiment 37. The disinfection robot of embodiment 22, wherein the disinfection robot further comprises a communication module configured to connect the robot to a remote computer system or external device, and receive instructions from the remote computer system or external device.

Embodiment 38. The disinfection robot of embodiment 22, wherein the disinfection robot is an autonomous robot and further comprises
(a) one or more motors configured to drive the wheels;
(b) one or more sensors configured to receive signals about the surrounding environment; and
(c) a navigation system configured to guide the robot to navigate autonomously.

Embodiment 39. The disinfection robot of embodiment 38, wherein the navigation system is a computer system configured to receive and process signals from the sensors, and generate one or more commands controlling the navigation and/or operation of the robot.

Embodiment 40. The disinfection robot of embodiment 38, wherein the robot has a tolerance distance of at least 20 mm on the left side from its center and at least 20 mm on the right side from its center.

Embodiment 41. The disinfection robot of embodiment 38, wherein the robot is capable of one or more of the following
(a) turning at the spot in 360 degree;
(b) steering around tight corners; and
(c) climbing to 15 degree.

Embodiment 42. An atomization system for converting a liquid into droplets, comprising
(a) a housing for holding at least one liquid;
(b) one or more atomizing elements configured to convert the liquid into droplets; and
(c) at least one outlet allowing the droplets to travel from the housing to the exterior or the housing.

Embodiment 43. The atomization system of embodiment 42, wherein the housing comprises one or more chambers for holding one or more type of disinfectants.

Embodiment 44. The atomization system of embodiment 42, wherein the atomization system converting a liquid into droplets by ultrasonic means, electrostatic means or a combination thereof.

Embodiment 45. The atomization system of embodiment 42, wherein the atomizing element is an ultrasonic transducer.

Embodiment 46. The atomization system of embodiment 42, wherein the atomizing element comprises an atomizing surface and a generator configured to cause vibration of the atomizing surface, wherein the liquid will be broken into droplets when it is in contact with the atomizing surface.

Embodiment 47. The atomization system of embodiment 42, wherein the atomizing element comprises a probe which the liquid may flow through, and a generator configured at the tip of the probe to cause vibration of the probe, wherein the liquid will be broken into droplets when it is in contact with tip of the probe.

Embodiment 48. The atomization system of embodiment 42, wherein the surface of the atomizing element is coated with a plurality of negative ions or charged particles.

Embodiment 49. The atomization system of embodiment 42, wherein the size of the droplets is in the range of 2 microns to 10 microns.

Embodiment 50. The atomization system of embodiment 42, wherein at least 95% of the population of the droplets generated has substantially the same size.

### Example 1—Disinfection of Model C-Train

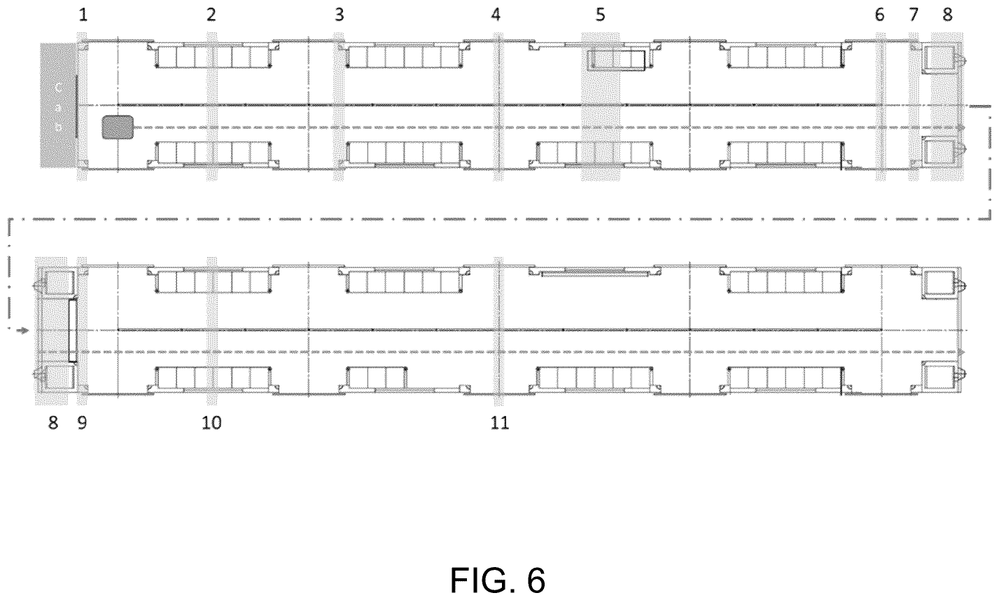

FIG. 6 shows a schematic diagram of a metro (model C-Train of the Hong Kong Mass Transit Railway system which represent a common interior design of all the trains of such system) where 11 cross-sections (cross-section 1 to 11) were treated and monitored during the disinfection process. The dimension of the metro is 24600 mm×3096 mm×3700 mm. The 11 cross-sections include various representative locations such as doors, seat, seat wall and gangway on the metro. Probes were placed at strategic locations on the cross-sections such as at rim area (e.g. on the wall) or blocked areas (e.g. under seat) to monitor the concentration of the disinfectant of the whole geometric plane.

In this embodiment, a mobile, automated disinfection robot, operated via remote control, was deployed at one end of the metro, and then dispersed vaporized hydrogen peroxide at each of the cross-sections as it moved along the metro towards the other end, with all the doors closed and ventilation turned off during the test. Vaporized hydrogen peroxide was applied by the robot at each of the 11 cross-section to achieve 90 ppm (parts per million) of hydrogen peroxide on the target cross-section (as detected by the probes located on those cross-sections) for at least 20 minutes. After the disinfection process was completed, all of the doors of the metro were opened for aeration for 30 minutes to reduce the disinfectant concentration to a level that is safe for human exposure. Bacterial level at the 11 cross-sections before and after treatment were monitored. It was found that keeping the vaporized hydrogen peroxide at 90 ppm for 20 minutes was able to reduce over 99.999% of Escherichia coli.

### Example 2—Disinfection of an Airport Express Line Train

To further evaluate the efficacy of disinfection of spaces on railway systems, a systematic field test was conducted on an Airport Express Line (AEL) train of the Mass Transit Railway (MTR) in Hong Kong using three different types of disinfectant composition. In this example, a mobile, automated disinfection robot that was operated via remote control was deployed at one end of the train, and then dispersed a disinfectant as it moved along the train towards the other end, with all the doors closed and ventilation turned off during the test. Table 1 summarizes the type of disinfectants and operating parameters of the disinfection process adopted in this example. As used herein, disinfectant consumption refers to the amount of disinfectant composition in its application form expressed in the unit of millimeter (mL) per unit volume of the disinfectant space expressed in cubic meter (m³). By way of example, a disinfection consumption of 1 mL/m³ means in a space of 1 m³ (1 m×1 m×1 m), it requires spraying 1 mL of the disinfectant composition in its application form by a device or otherwise means for the entire space.

TABLE 1
Summary of disinfectants and operating parameters in one
embodiment

| | EVICATE Pro | | NANOCYN | | BIOEM | |
|---|---|---|---|---|---|---|
| Active Ingredient | Quat | | Hypochlorous acid | | Plant extract | |
| Spray rate | 5 | L/hr | 5 | L/hr | 5 | L/hr |
| Speed setting | 0.1-0.2 | m/s | 0.1-0.2 | m/s | 0.1-0.2 | m/s |
| Disinfectant | 2.5 | mL/m³ | 3.2 | mL/m³ | 3.5 | mL/m³ |
| consumption | | | | | | |
| Concentration of | 10-40 | ppm | 10-40 | ppm | N.A. | |
| active ingredient | | | | | | |
| Particle size | 15-40 | μm | 15-40 | μm | 15-40 | μm |
| Contact time | 30 | min | 30 | min | 30 | min |
| Ventilation time | 10 | min | 10 | min | 10 | min |

Efficacy of disinfection was assessed by monitoring the surface cleanliness of the disinfected space using ATP bioluminescence assay. Adenosine triphosphate (ATP) is a measure of biological loading and used as a marker for bio-contamination to indicate the overall surface cleanliness. When there is a change (increase/decrease) in biomass, there is a corresponding change in ATP. The relative amount of ATP can then be measured with a portable device such as a 3M™ Clean-Trace™ Luminometer LX25 or 3M™ Clean-Trace™ Hygiene Monitoring Device and the result is expressed in relative light unit (RLU). In the context of disinfection, surface cleanliness monitoring test serves as a quick on-site test to evaluate if a cleaning and disinfection process has been carried out satisfactorily by reference to the cell viability or bioburden. Generally, the lower the RLU reading, the smaller amount of microorganism is in the sample and the cleaner is the surface. In healthcare settings, the recommended acceptance criteria of RLU are as follows: a) Pass (<500 RLU), b) Caution (500-1,000 RLU), and c) Fail (>1,000 RLU).

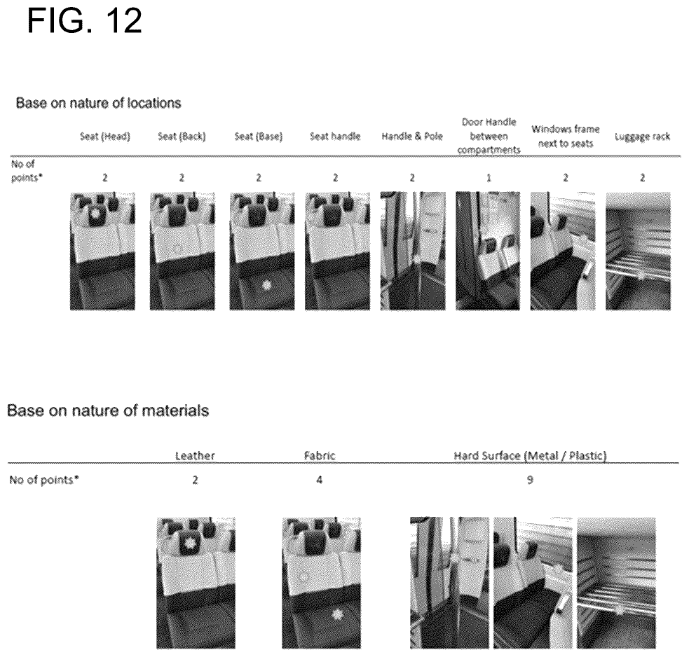

In this example, two samples were collected from each of the target areas on the train by two independent swapping (sample area of 2 cm×5 cm) immediately before and after the disinfection and their respective RLU readings were measured using a 3M™ Clean-Trace™ Hygiene Monitoring Device. As shown in FIG. 12, a total of 11 sample locations covering various touchable areas and materials on the train were selected and tested. Tables 2-4 provide a summary of the results of the surface cleanliness monitoring test of this example.

TABLE 2
Summary of results of surface cleanliness monitoring test of a train using EVICATE Pro
EVICATE Pro

| | | Before Disinfection | | After Disinfection | | |
|---|---|---|---|---|---|---|
| | | (RLU Readings) | | (RLU Readings) | | % |
| | | Average | Range | Average | Range | Reduction |
| Location | Seat (Head) | 99 | 89-108 | 25 | 15-34 | 75 |
| | Seat (Back) | 219 | 105-333 | 33 | 31-35 | 85 |
| | Seat (Base) | 391 | 162-620 | 41 | 36-46 | 90 |
| | Seat | 137 | 73-201 | 30 | 23-37 | 78 |
| | Handle | | | | | |
| | Handle & | 162 | 53-270 | 12 | 9-14 | 93 |
| | Pole | | | | | |
| | Door | 19 | N.A. | 6 | N.A. | 68 |
| | Handle | | | | | |
| | Windows | 58 | 58-58 | 22 | 22-22 | 62 |
| | Frame | | | | | |
| | Luggage | 164 | 18-310 | 14 | 7-21 | 91 |
| | Rack | | | | | |
| Materials | Leather | 99 | 89-108 | 25 | 15-34 | 75 |
| | Fabric | 58 | 105-620 | 37 | 31-46 | 88 |
| | Hard | 125 | 18-310 | 17 | 6-37 | 86 |
| | Surface | | | | | |
| | All Points | 173 | 18-620 | 24 | 6-46 | 80 |

TABLE 3
Summary of results of surface cleanliness monitoring test of a train using NANOCYN
NANOCYN

| | | Before Disinfection | | After Disinfection | | |
|---|---|---|---|---|---|---|
| | | (RLU Readings) | | (RLU Readings) | | % |
| | | Average | Range | Average | Range | Reduction |
| Location | Seat (Head) | 227 | 192-262 | 44 | 25-62 | 81 |
| | Seat (Back) | 162 | 61-262 | 47 | 23-70 | 71 |
| | Seat (Base) | 358 | 196-520 | 60 | 57-62 | 83 |
| | Seat | 124 | 28-220 | 40 | 21-59 | 68 |
| | Handle | | | | | |
| | Handle & | 47 | 39-54 | 17 | 11-23 | 63 |
| | Pole | | | | | |
| | Door | 53 | N.A. | 28 | N.A. | 47 |
| | Handle | | | | | |
| | Windows | 43 | 43-43 | 11 | 11-11 | 74 |
| | Frame | | | | | |
| | Luggage | 100 | 78-121 | 27 | 21-33 | 73 |
| | Rack | | | | | |
| Materials | Leather | 227 | 192-262 | 44 | 25-62 | 81 |
| | Fabric | 260 | 61-520 | 53 | 23-70 | 80 |
| | Hard | 80 | 28-220 | 26 | 11-59 | 67 |
| | Surface | | | | | |
| | All Points | 150 | 28-520 | 36 | 11-70 | 70 |

TABLE 4
Summary of results of surface cleanliness monitoring test of a train using BIOEM
BIOEM

| | | Before Disinfection | | After Disinfection | | |
|---|---|---|---|---|---|---|
| | | (RLU Readings) | | (RLU Readings) | | % |
| | | Average | Range | Average | Range | Reduction |
| Location | Seat (Head) | 93 | 68-118 | 75 | 58-91 | 20 |
| | Seat (Back) | 270 | 90-450 | 249 | 86-411 | 8 |
| | Seat (Base) | 272 | 163-380 | 246 | 121-371 | 9 |
| | Seat | 48 | 18-77 | 23 | 8-37 | 53 |
| | Handle | | | | | |
| | Handle & | 103 | 89-116 | 49 | 26-71 | 53 |
| | Pole | | | | | |
| | Door | 56 | N.A. | 45 | N.A. | 20 |
| | Handle | | | | | |
| | Windows | 130 | 130-130 | 81 | 81-81 | 38 |
| | Frame | | | | | |
| | Luggage | 29 | 28-29 | 19 | 15-23 | 33 |
| | Rack | | | | | |
| Materials | Leather | 93 | 68-118 | 75 | 58-91 | 20 |
| | Fabric | 271 | 90-450 | 247 | 86-411 | 9 |
| | Hard | 68 | 18-130 | 38 | 8-81 | 44 |
| | Surface | | | | | |
| | All Points | 129 | 18-450 | 103 | 8-411 | 29 |

The results indicated that the disinfection using EVICATE Pro or NANOCYN considerably improved the surface cleanliness of all the selected locations passing the while the disinfection using BIOEM was less effective.

As one skilled in the art will appreciate, the present method can be used to disinfect railway systems of various designs and is not limited to the type of trains illustrated or described herein. In some embodiments, the present method can be used to disinfect train cabins of the Mass Transit Railway (MTR) in Hong Kong or the mainland of China, such as Metro Cammell EMU (DC) (M-train), Adtranz-CAF EMU, Rotem EMU, CNR Changchun EMU (C-train), SP1900/1950 EMU, Metro Cammell EMU (AC), Hyundai Rotem EMU (R-stock) and light rail rolling stock. In some embodiments, the present method can be used to disinfect train cabins of high speed trains such as those operated in the mainland of China or Hong Kong including CRH2 Class, CRH2A Class, CRH2 Class x2 & Passenger cars, CRH3 Class, CRH3 Class, CRH380AL Class, CRH400AF Class, CRH400AF Class, CRH5 Class, CRH5 Class x2 & Passenger cars, DJJ Class “Blue Arrow” Train, X-2000 Tilt Train, and KCR Metropolitan Train (Hong Kong).

As one skilled in the art will appreciate, no matter a manual, stationary or mobile device is employed for dispersing the disinfectant droplets to the space, it is important to spray sufficient amount of disinfectant to achieve an effective concentration throughout the metro (especially at the blocked or less exposed areas) to ensure sufficient coverage and allow sufficient time for the reaction to happen. The treatment time may vary according to the type and quantity of the target microorganism in question, the type of disinfectant used and the pre-treatment conditions of metro cabin. In some embodiments, to disinfect a metro cabin of about 9,000-11,000 cubic feet, the effective concentration of the hydrogen peroxide vapours within the cabin or on the target surfaces is in the range of 120-400 ppm, and the treatment time is in the range of 20-360 minutes. In some other embodiments, to disinfect a metro cabin of about 9,000-11,000 cubic feet, the effective concentration of the quat-based disinfectant droplets within the cabin or on the target surfaces is in the range of 10-40 ppm, and the treatment time is in the range of 20-360 minutes. In some other embodiments, to disinfect a metro cabin of about 9,000-11,000 cubic feet, the effective concentration of the hypochlorous acid-based disinfectant droplets within the cabin or on the target surfaces is in the range of 10-40 ppm, and the treatment time is in the range of 20-360 minutes.

In some other embodiments, the method described herein reduces the quantity, cell viability and/or infectivity of a target microorganism in a confined space on a railway system and reduces the bioburden of the target microorganism by 90.0-99.9% within a period of time. In yet other embodiments, the method described herein reduces the RLU reading of a confined space on a railway system by at least 70%, 75%, 80%, 85%, 90%, or 95% within a period of time.

### Disinfection of Aircraft Cabins

In one aspect, there is provided a method of disinfecting a space on an aircraft cabin. In yet another aspect, there is provided a method of reducing the quantity, cell viability and/or infectivity of a target microorganism in a space on an aircraft cabin.

The method comprises the steps of a) dispersing into a space on an aircraft cabin a multiplicity of droplets of disinfectant composition, and b) allowing a time sufficient for the droplets to distribute throughout the space thereby maintaining an effective concentration of the disinfectant composition within the space for a period of time. As described herein, the droplets can be dispersed manually or by automated means, and various types of disinfectant may be used.

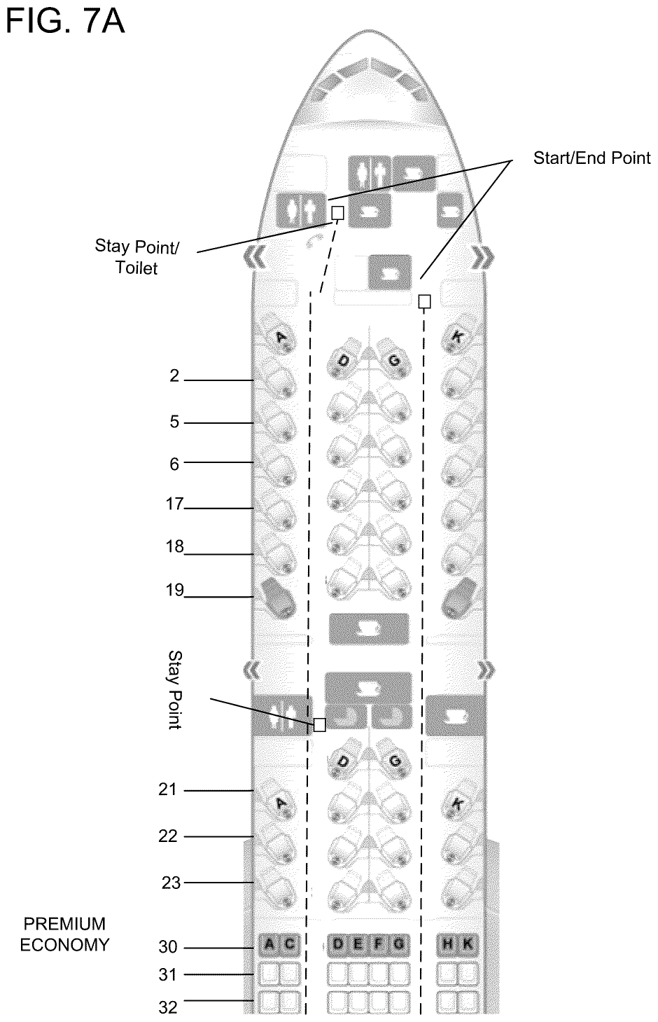

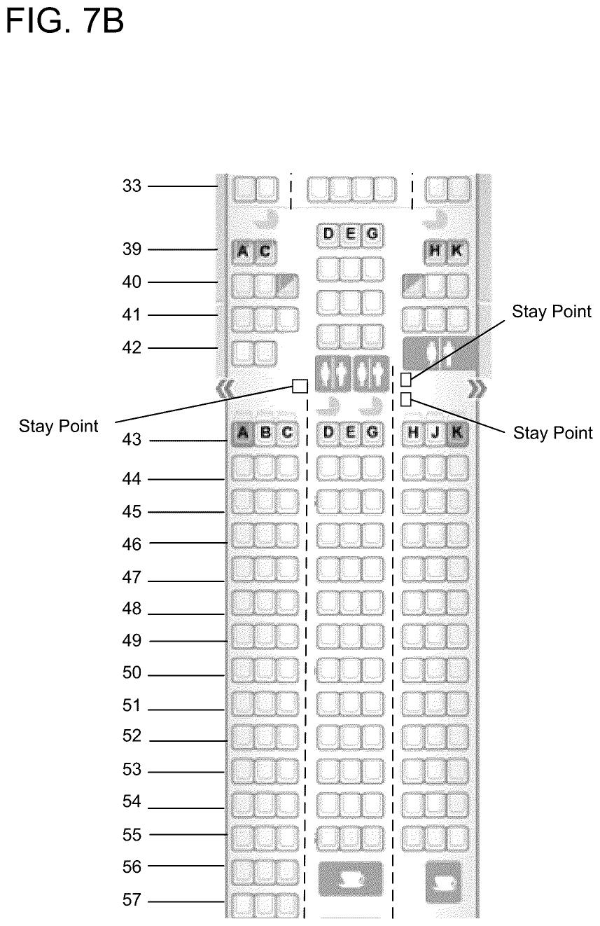

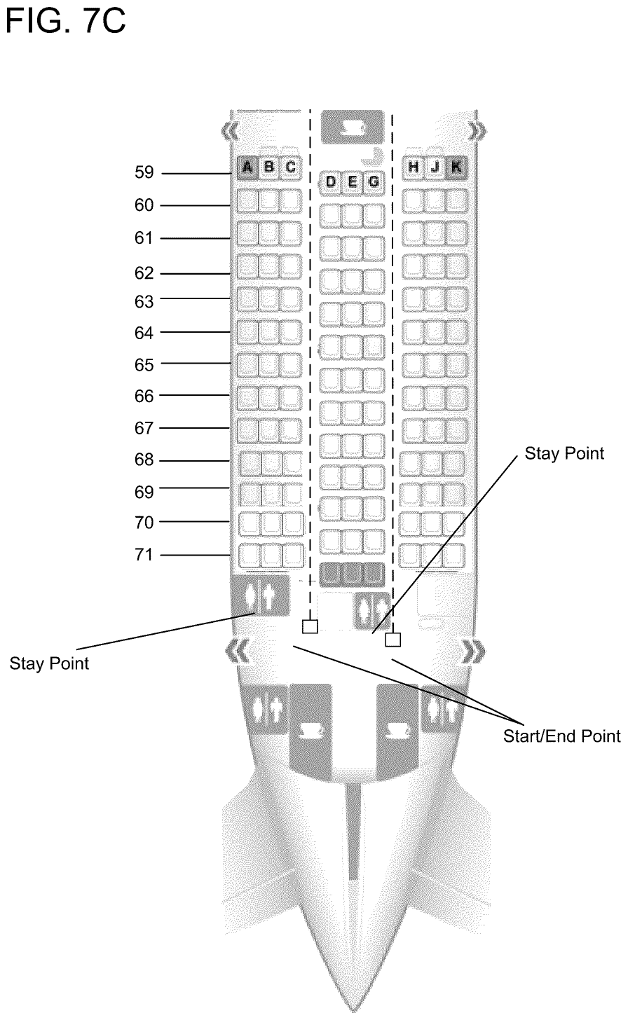

FIG. 7 shows a schematic diagram of an aircraft cabin with two aisles. In one embodiment, one mobile, automated disinfection robot is placed on each aisle at the start point, disperses disinfectant droplets while moving along the aisle till the other end of the aircraft, with all the doors and windows of the aircraft closed and all ventilating devices including the air conditioning turned off. The two robots are configured to stop at each of the stay points (which are the partition between cabin classes or toilet area) for a period of time such as 15 seconds. The total disinfection was about 10 to 15 minutes. After the disinfection process is completed, all of the doors of the aircraft are opened for aeration to reduce the disinfectant concentration to a level that is safe for human exposure. Level of the target microorganism at selected locations within the cabin before and after treatment are monitored. It is expected that the present method is capable of significantly reducing the amount of target microorganism and be satisfactory on keeping a low bio-burden.

As one skilled in the art will appreciate, no matter a manual, stationary or mobile device is employed for dispersing the disinfectant droplets, it is important to spray sufficient amount of disinfectant to achieve an effective concentration throughout the aircraft cabin (especially at the blocked or less exposed areas) to ensure sufficient coverage and allow sufficient time for the reaction to happen. The treatment time may vary according to the type and quantity of the target microorganism in question, the type of disinfectant used and the pre-treatment conditions of the aircraft cabin.

The present method can be used on various types of aircraft, no matter passenger aircrafts or cargo aircrafts, including without limitation BOEING aircrafts (e.g. models 707, 720, 727, 737, 747, 757, 767, 777 and 797), and AIRBUS aircrafts (e.g. models A220, A300, A310, A318, A319, A320, A321, A330, A340, A350 and A380).

In some embodiments, to disinfect an aircraft cabin of BOEING model 737, the effective concentration of the quat-based disinfectant droplets in the cabin is in the range of 10-40 ppm, and the treatment time is in the range of 20-360 minutes. In some other embodiments, to disinfect an aircraft cabin of AIRBUS model A320, the effective concentration of the quat-based disinfectant fog or mist droplets in the cabin is in the range of 10-40 ppm, and the treatment time is in the range of 20-360 minutes.

In various embodiments, the disinfectants that can be used in couple with the present invention are those in compliance with the aerospace industry standards or other applicable standards adopted by companies operating in the aerospace industry the such as “BOEING SPECIFICATION STANDARD BSS7434” for testing the chemical compatibility of cleaning products and interiors parts/materials of commercial transport aircraft and “AIRBUS INDUSTRIES BRITISH AEROSPACE AIRBUS AIMS09-00-002” which evaluates the competently of maintenance materials with Airbus.

In one embodiments, the disinfectant to be used in couple with the present invention is NANOCYN by MICROSAFE, a hypochlorous acid-based disinfectant that is able to eliminate a wide range of viruses (e.g. Herpes Simplex, Norovirus (Gastro), Influenza A (H1N1), Coronavirus, including SARS-CoV-2 (virus that causes COVID-19), Hepatitis A and B), bacteria (e.g. Methicillin-resistant Staphylococcus aureus (MRSA), Escherichia coli, Pseudomonas aeruginosa), mold and fungi (e.g. Candida Albicans, Trichophyton mentagrophytes) and other microorganisms with a 6-log reduction (i.e. 99.9999% reduction) in 30 second contact time. NANOCYN/MICROSAFE has been approved for disinfection of certain surfaces and air-spaces by way of spraying or fogging (also known as fumigation or misting) by the regulatory authority in the Middle East/Africa, Europe, Australia and other regions, and will be approved in the United States soon.

In one embodiment, the present invention provides a method of disinfecting an aircraft cabin, comprising the steps of a) dispersing into a space on an aircraft cabin a multiplicity of droplets of a hypochlorous acid-based disinfectant solution, and b) allowing a time sufficient for the droplets to distribute throughout the space thereby maintaining an effective concentration of the disinfectant composition within the space for a period of time. In some embodiments, the effective concentration of the hypochlorous acid droplets is in the range of 10-200 ppm, and the treatment time is in the range of 15-240 minutes.

### Example 3—Disinfection of AIRBUS A350 Cabin

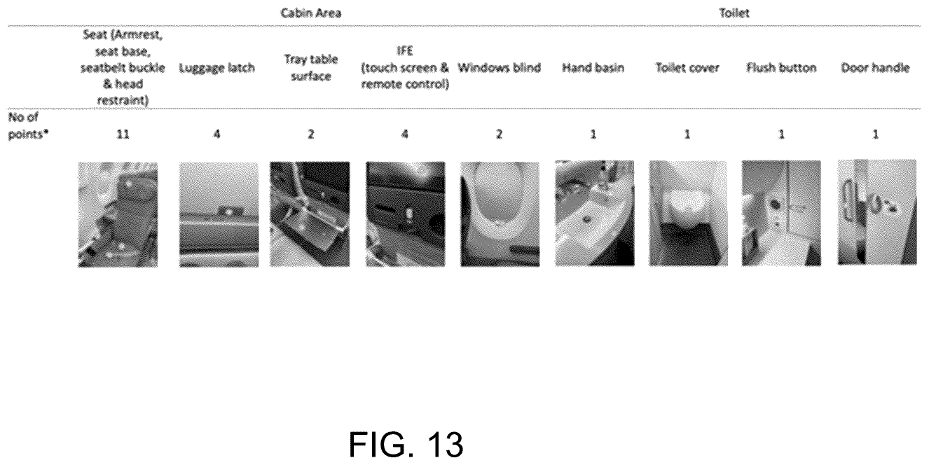

In one embodiment, the present invention provides a method of disinfecting a flight cabin (model AIRBUS A350) using a disinfection robot. Surface cleanliness test was conducted to evaluate the efficacy of the disinfection. With reference to FIG. 7A to FIG. 7C, a disinfection robot was placed on each aisle at the start point, dispersed droplets of NANOCYN while moving along the aisle till the other end of the aircraft, with all the doors and windows of the aircraft closed and all ventilating devices including the air conditioning turned off. The two robots were configured to stop at each of the stay points (which are the partition between cabin classes or toilet area) for 15-30 seconds. The total disinfection time was about 15-30 minutes. The concentration of NANOCYN within the flight cabin is about 10-40 ppm. Samples at selected locations as shown in FIG. 13 within the cabin before and after treatment were collected and tested using the surface cleanliness test.

Table 5 summarizes the results which indicated that the present invention using NANOCYN was able to achieve satisfactory disinfection results on most of the tested locations as seen by a significant drop in the RLU readings.

TABLE 5
Summary of results of surface cleanliness monitoring
test of a flight cabin using NANOCYN
NANOCYN

| | | Before Disinfection | | After Disinfection | | |
|---|---|---|---|---|---|---|
| | | (RLU Readings) | | (RLU Readings) | | % |
| | | Average | Range | Average | Range | Reduction |
| Location | Armrest | 618 | 172-1462 | 32 | 13-65 | 95 |
| | Seat Belt | 166 | 76-255 | 38 | 12-64 | 77 |
| | Buckle | | | | | |
| | Head | 814 | 763-865 | 45 | 36-54 | 94 |
| | Restraint | | | | | |
| | Seat Base | 433 | 344-522 | 87 | 45-129 | 80 |
| | Latch | 24 | 6-23 | 16 | 6-21 | 31 |
| | Tray Table | 290 | 30-35 | 10 | 17-21 | 97 |
| | Surface | | | | | |
| | IFE Remote | 54 | 47-61 | 45 | 41-48 | 18 |
| | Control | | | | | |
| | IFE Touch | 9 | 6-11 | 8 | 4-11 | 12 |
| | Screen | | | | | |
| | Windows | 72 | 38-105 | 10 | 8-12 | 86 |
| | Blind | | | | | |
| | Seat Pocket | 145 | 53-236 | 33 | 20-45 | 78 |
| | Hand Basin | 780 | N.A. | 48 | N.A. | 94 |
| | Toilet Cover | 208 | N.A. | 17 | N.A. | 92 |
| | Flush | 158 | N.A. | 6 | N.A. | 96 |
| | Button | | | | | |
| | Door | 115 | N.A. | 27 | N.A. | 77 |
| | Handle | | | | | |
| Materials | Leather | 814 | 763-865 | 45 | 36-54 | 94 |
| | Fabric | 289 | 53-522 | 60 | 20-129 | 79 |
| | Hard | 206 | 6-1,462 | 23 | 4-65 | 89 |
| | Surface | | | | | |

In one embodiment, there is provided a method of reducing the quantity, cell viability and/or infectivity of a target microorganism in an aircraft cabin, the method comprises a step of dispersing into the aircraft cabin a multiplicity of droplets of a disinfectant composition, and a step of allowing a time sufficient for the droplets to distribute throughout the aircraft cabin thereby maintaining an effective concentration of the disinfectant composition in the aircraft cabin for a period of time.

In one embodiment, the effective concentration is a concentration of the disinfectant composition in the aircraft cabin that is sufficient to reduce the bioburden of the target microorganism in the aircraft cabin by 90.0%-99.9% within a period of time.

In one embodiment, the present method of reducing the quantity, cell viability and/or infectivity of a target microorganism in an aircraft cabin comprises dispersing to the cabin of a BOEING aircraft droplets of a disinfectant composition in accordance with the following parameters:

| | | Con- | | | Average |
|---|---|---|---|---|---|
| | | centration | | | reduction |
| | | of active | | | of |
| | | ingredient | Aver- | Airborne | bacterial |
| | Examples | within the | age | dis- | level |
| | of | flight | droplet | infectant | after |
| Active | disinfectant | cabin | size | contact | treatment |
| ingredient | composition | (ppm) | (μm) | time (min) | (%) |
| Hypochlorous | NANOCYN | 10-40 | 15-40 | 15-30 | 90-99.9 |
| acid | | | | | |

In one embodiment, the present method of reducing the quantity, cell viability and/or infectivity of a target microorganism in an aircraft cabin comprises dispersing droplets to the cabin of an AIRBUS aircraft of a disinfectant composition containing the following active ingredients and in accordance with the following parameters:

| | | Con- | | | Average |
|---|---|---|---|---|---|
| | | centration | | | reduction |
| | | of active | | | of |
| | | ingredient | Aver- | Airborne | bacterial |
| | Examples | within the | age | dis- | level |
| | of | flight | droplet | infectant | after |
| Active | disinfectant | cabin | size | contact | treatment |
| ingredient | composition | (ppm) | (μm) | time (min) | (%) |
| Hypochlorous | NANOCYN | 10-40 | 15-40 | 15-30 | 90-99.9 |
| acid | | | | | |

In some embodiments, the method described herein reduces the quantity, cell viability and/or infectivity of a target microorganism in a confined space on an aircraft cabin and reduces the bioburden of the target microorganism by 90.0-99.9% within a period of time. In yet other embodiments, the method described herein reduces the RLU reading of a confined space on an aircraft cabin reading by at least 70%, 75%, 80%, 85%, 90%, or 95% within a period of time.

### Disinfection of Confined Rooms

In one aspect, there is provided a method of disinfecting a confined room such as a hotel room, a hostel room and a room in a workplace or school. In yet another aspect, there is provided a method of reducing the quantity, cell viability and/or infectivity of a target microorganism in a confined area. Other rooms such as those in hospitals, aged care centers, child care centers and the like can also be treated by the present methods. The method comprises the steps of a) dispersing into a room a multiplicity of droplets of a disinfectant composition, and b) allowing a time sufficient for the droplets to distribute throughout the room thereby maintaining an effective concentration of the disinfectant composition within the room for a period of time. As described herein, the droplets can be dispersed manually or by automated means, and various types of disinfectant may be used.

### Example 4—Disinfection of an Enclosed Carpeted Conference Room

FIG. 8 shows a floor plan of an enclosed carpeted conference room of about 200 square feet and 8 feet in height, without any partition from ceiling to floor. Bacteria were placed in 10 locations (locations A-J) around the room. A stationary disinfection device was deployed at one end of the table (location J) and configure to disperse droplets of a quat-based disinfectant thereto for 5 minutes at room temperature and standard atmospheric pressure, with all the doors and windows of the room closed and all ventilating devices including the air conditioning turned off. After the application of the droplets, the room was then left undisturbed for at least 30 minutes, and bacterial samples were collected from locations A-J after the whole process. Bacterial level at locations A-J before and after the process were measured and compared (Table 6). It was found that on average about 90%-99.99% of carrier bacteria were been eliminated after the treatment.

TABLE 6
Reduction of bacterial level at locations A-J after
treatment of a confined room

| Locations | Reduction of bacterial level after treatment (%) |
|---|---|
| A | 99.82 |
| B | 99.79 |
| C | 99.83 |
| D | 99.59 |
| E | 99.90 |
| F | 96.81 |
| G | 96.40 |
| H | 98.79 |
| I | 99.99 |
| J | 99.83 |

### Example 5—Disinfection of an Enclosed Non-Carpeted Room

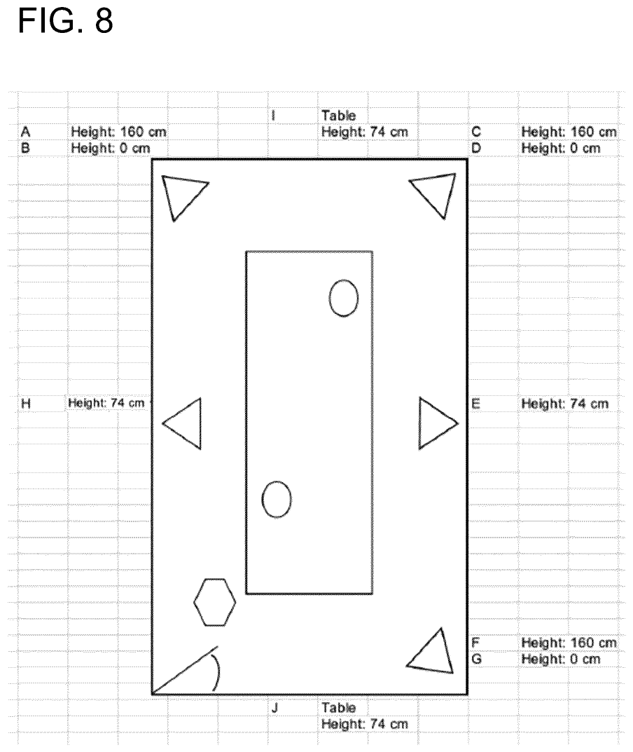

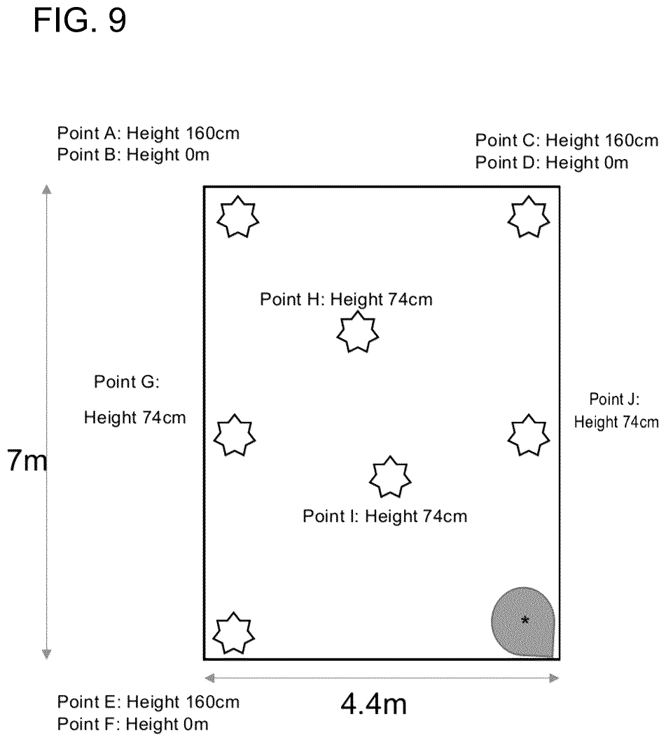

FIG. 9 shows a floor plan of an enclosed non-carpeted room of about 320 square feet and 9 feet in height, without any partition from ceiling to floor. Bacteria were placed in locations A-J around the room. A stationary disinfection device was deployed at one of the corners of the room (as marked *) and configured to disperse droplets of disinfectant thereto for 18 minutes, followed by a waiting period for 30 minutes. The whole process was conducted at room temperature and standard atmospheric pressure, with all the doors and windows of the room closed and all ventilating devices including the air conditioning turned off. A series of tests using various strains of bacteria was performed, and their levels before and after treatment were monitored. Table 7 summarizes the types of disinfectant used and the average reduction of bacterial level after treatment.

TABLE 7
Reduction of bacterial level by treating a confined room by various
types of disinfectants

| | | | Average reduction |
|---|---|---|---|
| | Active | Solution | of bacterial level |
| Disinfectants | ingredients | concentration | after treatment (%) |
| NANOCYN | Hypochlorous acid | Use as served | 50-90 |
| ECOLAB | Quat | 1:49 (v/v) | >99 |
| DESGUARD 20 | | | |
| EVICATE Pro | Quat | Use as served | 50-99 |
| DIVERSEY | Quat | 1:256 (v/v) | 50-80 |
| TERSANO | Ozone | User as served | 50-80 |

### Example 6—Disinfection of an Enclosed Room Using a Quat-Based Disinfectant

FIG. 10 shows a floor plan of an enclosed room of about 320 square feet and 498 cm (about 16.34 feet) in height, where bacteria were placed on selected locations in the room including those on the walls (W1-W4), corners (C1-C4) and table (T1-T3) at same or different heights. A mobile, automated disinfection device was deployed and configured to move within the room following a pre-determined route as indicated by the arrow route twice and disperse droplets of a quat-based disinfectant (active ingredient concentration: 0.05%-1% (wt/wt)) with a total spraying time of about 4 minutes. The room was then left undisturbed for 2 hours. The whole process was conducted at room temperature and standard atmospheric pressure, with all the doors and windows of the room closed and all ventilating devices including the air conditioning turned off.

Table 8 summarizes the average reduction of bacterial level after treatment, showing that the treatment was able to reduce >99% of the two tested bacterial strains in all selected locations and heights.

TABLE 8

| Reduction of bacterial level by treating a confined room | |
|---|---|
| with a quat-based disinfectant using a mobile robot | |
| | Average reduction of |
| | bacterial level after |
| Microorganism | treatment (%) |
| Escherichia coli | >99 |
| Staphylococcus aureus | >99 |
| | Average reduction of |
| | bacterial level after |
| Location | treatment (%) |
| Wall (W1-W4) | >99 |
| Corner (C1-C4) | >99 |
| Table (T1-T3) | >99 |
| | Average reduction of |
| | bacterial level after |
| Height | treatment (%) |
| 0 mm | >99 |
| 800 mm | >99 |
| 1500 mm | >99 |

### Example 7—Disinfection of an Enclosed Room Using NANOCYN

In this example, a simulation test was conducted in an enclosed room of about 330 square feet by an accredited laboratory using microbiology method to evaluate the disinfection efficacies of NANOCYN dispersed by one embodiment of the disinfection robot described herein.

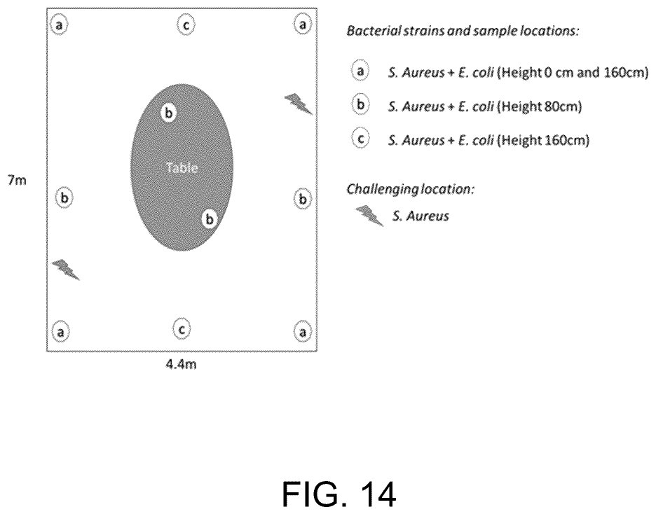

FIG. 14 illustrates an exemplary floor plan of an enclosed room of about 330 square feet and 300 cm (about 9.8 feet) in height, where S. aureau and/or E. coli were placed on selected locations in the room. A mobile, automated disinfection device was deployed and configured to move within the room following a pre-determined route and disperse droplets of NANOCYN with a total spraying time of about 2-10 minutes. The room was then left undisturbed for about 1-6 hours. The whole process was conducted at room temperature and standard atmospheric pressure, with all the doors and windows of the room closed and all ventilating devices including the air conditioning turned off. It is estimated the concentration of NANOCYN within the room was maintained at about 10-40 ppm during the treatment period.

In this simulation test, it was found that bacterial levels at all selected locations were reduced over 90%.

### Example 8—Disinfection of an Enclosed Room Using EVICATE Pro and NANOCYN

In this example, a simulation test was conducted in an enclosed room of about 200 square feet and 300 cm (about 9.8 feet) in height, by an accredited laboratory using microbiology method to evaluate the disinfection efficacies of EVICATE Pro and NANOCYN dispersed by a mobile automated disinfection robot under three different disinfection protocols:
1. Protocol I (precautionary disinfection) which may be adopted for routine disinfection of low-risk areas.
2. Protocol II (mid-level disinfection) which may be adopted for routine disinfection of high-risk areas, or disinfection of moderately contaminated areas.
3. Protocol III (high-level disinfection) which may be adopted for routine disinfection of high-risk areas, or disinfection of heavily contaminated areas.

The pathogen tested was S. aureus and E. coli. Key operating parameters and results are summarized in Table 9 and Table 10.

TABLE 9

| Summary | | | | | | |
|---|---|---|---|---|---|---|
| of a simulated | | | | | | |
| testing using | | | | | | |
| EVICATE Pro to | Protocol I | | Protocol II | | Protocol III | |
| disinfect a confined | (precautionary | | (mid-level | | (high-level | |
| room | disinfection) | | disinfection) | | disinfection) | |
| Spray rate | 2 | L/hr | 2 | L/hr | 2 | L/hr |
| Speed setting | 0.1-1 | m/s | 0.1-1 | m/s | 0.1-1 | m/s |
| Disinfectant | 1 | mL/m³ | 2 | mL/m³ | 6 | mL/m³ |
| Consumption | | | | | | |
| Concentration of | 1-5 | ppm | 1-10 | ppm | 10-40 | ppm |
| active ingredient | | | | | | |
| Airborne disinfection | 2 | hours | 4 | hours | 6 | hours |

| | | | |
|---|---|---|---|
| contact time | | | |
| Reduction of pathogen | >90% | >99% | >99.9% |

TABLE 10
Summary of a simulated testing using NANOCYN to disinfect a
confined room

| | Protocol I | | Protocol II | | Protocol III | |
|---|---|---|---|---|---|---|
| | (precautionary | | (mid-level | | (high-level | |
| | disinfection) | | disinfection) | | disinfection) | |
| Spray rate | 2 | L/hr | 2 | L/hr | 2 | L/hr |
| Speed setting | 0.1-1 | m/s | 0.1-1 | m/s | 0.1-1 | m/s |
| Disinfectant | 2 | mL/m³ | 4 | mL/m³ | 6 | mL/m³ |
| Consumption | | | | | | |
| Concentration of | 1-5 | ppm | 1-10 | ppm | 10-40 | ppm |
| active ingredient | | | | | | |
| Airborne disinfection | 2 | hours | 4 | hours | 6 | hours |
| contact time | | | | | | |

| | | | |
|---|---|---|---|
| Reduction of pathogen | >90% | >99% | >99.9% |

### Example 9—Disinfection of an Elderly Home

In this example, an full-depth disinfection in high-risk areas and precautionary disinfection in common areas in an elderly home was conducted using the present invention. In brief, each selected area was disinfected for a period of time using a mobile disinfection robot dispersing EVICATE Pro and a manual-operated disinfection robot dispersing EVICATE Pro sequentially. For high-risk areas (such as toilet, pantry, kitchen, store room and multifunction room), the disinfectant consumption was about 4-8 mL/m³. For common areas (such as dining room, residential compartments and corridors), the disinfectant consumption was about 6-10 mL/m³. The room was then left undisturbed for 2 hours after treatment and ventilated before the reentering by residents and staffs. Disinfection efficacies were evaluated by the surface cleanliness tested described above.

Table 11 summarizes the results of precautionary disinfection of the common areas.

TABLE 11
Summary of results of surface cleanliness monitoring test of selected
areas subject to precaution disinfection using EVICATE Pro

| | | Before Disinfection | | After Disinfection | | |
|---|---|---|---|---|---|---|
| Sampling Locations (no. | | (RLU Readings) | | (RLU Readings) | | % |
| of sampling points) | | Average | Range | Average | Range | Reduction |
| Living and | Table | 9,861 | 5,512-15,403 | 456 | 380-491 | 95 |
| dining zone | surface | | | | | |
| | (4) | | | | | |
| | Chair arm | 1,031 | 761-1,533 | 143 | 73-185 | 86 |
| | test (4) | | | | | |
| | Chair arm | 2,045 | 1462-2,627 | 274 | 191-357 | 87 |
| | rest on | | | | | |
| | living area | | | | | |
| | (2) | | | | | |
| Residential | Table | 891 | 80-1,995 | 97 | 12-311 | 89 |
| compartments | surface | | | | | |
| | (4) | | | | | |
| | Bed | 584 | 190-1,283 | 125 | 15-381 | 79 |
| | handle (4) | | | | | |
| Corridors | Door | 2,784 | 352-8,075 | 129 | 18-411 | 95 |
| | handle | | | | | |

The results indicate a consistent and significant drop of RLU in every selected locations upon disinfection process, indicating an improvement on overall hygiene. Furthermore, the surface cleanliness passed the acceptance criteria on the guide of general health care settings.

### Example 10—Disinfection of Rooms in a Hotel

In this example, a field test was conducted in a hotel to evaluate the efficacies of using the present invention to disinfect selected areas within a hotel. A total of 36 areas were selected among the ball room, guest floor, pre-occupied guest room and housekeeping office and disinfected using a mobile disinfection robot dispersing NANOCYN. The disinfectant consumption was about 1-2 mL/m³. Each of the treated areas was then left undisturbed for about 30 minutes after treatment. For disinfection of carpets, vacuum cleaning was performed prior to the disinfection to physically remove the dirt and microbes that was hidden in the textile fibers of the carpets in order to improve the effectiveness of the disinfection.

Disinfection efficacies were evaluated by the surface cleanliness tested described above.

Tables 12-15 summarize the results of disinfection of these selected areas.

TABLE 12
Summary of results of surface cleanliness monitoring test of selected
areas in a ball room and a reception in a hotel using NANOCYN

| | | Before | After | |
|---|---|---|---|---|
| | | Disinfection | Disinfection | |
| | | (RLU | (RLU | % |
| Sampling Locations | | Reading) | Reading) | Reduction |
| Reception | Carpet I | 1,506 | 165 | 89 |
| of Ball | Carpet II | 724 | 166 | 77 |
| Room | Door Handle I | 955 | 9 | 99 |
| | Door Handle II | 490 | 97 | 80 |
| | Bar Table | 48 | 35 | 27 |
| Ball | Chair I | 652 | 67 | 90 |
| Room | Chair II | 1,709 | 112 | 93 |
| | Table I | 312 | 64 | 79 |

| | | | |
|---|---|---|---|
| Average | 800 | 89 | 89 |

TABLE 13
Summary of results of surface cleanliness monitoring test of
selected areas on a guest floor in a hotel using NANOCYN

| | | Before | After | |
|---|---|---|---|---|
| | | Disinfection | Disinfection | |
| | | (RLU | (RLU | % |
| Sampling Locations | | Reading) | Reading) | Reduction |
| Guest | Lift Button | 174 | 22 | 87 |
| Floor | | | | |
| | Phone | 187 | 23 | 88 |
| | Carpet I | 208 | 31 | 85 |
| | Carpet II | 261 | 23 | 91 |
| | Carpet III | 677 | 82 | 88 |
| | Carpet IV | 175 | 51 | 71 |
| | Door Handle I | 2,765 | 101 | 96 |
| | Door Handle II | 834 | 194 | 77 |

| | | | |
|---|---|---|---|
| Average | 660 | 66 | 90 |

TABLE 14
Summary of results of surface cleanliness monitoring test of selected
areas in a guest room in a hotel using NANOCYN

| | | Before | After | |
|---|---|---|---|---|
| | | Disinfection | Disinfection | |
| | | (RLU | (RLU | % |
| Sampling Locations | | Reading) | Reading) | Reduction |
| Guest | Footstools | 2,335 | 373 | 84 |
| Room | | | | |
| | Remote | 400 | 169 | 58 |
| | Coffee Table | 419 | 48 | 89 |
| | Desk | 465 | 46 | 90 |
| | Pillows | 525 | 286 | 46 |
| | Chair | 831 | 46 | 94 |
| | Bedside table | 525 | 48 | 91 |
| | Phone | 295 | 33 | 89 |
| | Sofa | 1,216 | 93 | 92 |
| Bathroom | Basin | 2,401 | 113 | 95 |
| in Guest | | | | |
| Room | | | | |
| | Toilet Cover | 183 | 48 | 74 |
| | Shelf | 161 | 26 | 84 |
| | Bidet | 319 | 55 | 83 |
| | Faucet | 11,015 | 700 | 83 |

| | | | |
|---|---|---|---|
| Average | 1,506 | 149 | 90 |

TABLE 15
Summary of results of surface cleanliness monitoring test of selected
areas in a housekeeping office in a hotel using NANOCYN

| | | Before | After | |
|---|---|---|---|---|
| | | Disinfection | Disinfection | |
| | | (RLU | (RLU | % |
| Sampling Locations | | Reading) | Reading) | Reduction |
| Housekeeping | Corridor Handle | 2,636 | 276 | 90 |
| Office | | | | |
| | Door Handle I | 1,424 | 916 | 36 |
| | Door Handle II | 148 | 74 | 50 |
| | Door | 297 | 77 | 74 |
| | Tray | 2,495 | 552 | 78 |
| | Lift Button | 1,595 | 436 | 73 |

| | | | |
|---|---|---|---|
| Average | 1,433 | 388 | 73 |

The results indicated that the average of the RLU of the 36 spots before disinfection was over 1000 RLU which was considered high. A consistent and significant drop in RLU readings was observed on every selected location upon disinfection process (>80% on average), with an average of 150 RLU. In particular, the average drop in RLU is 89% for the ball room and its reception area, 90% for the guest floor, 90% for the guest room and its bathroom, and 73% of the housekeeping office. Overall, the results suggest that a satisfactory improvement in surface cleanliness were achieved by the present invention and resulting surface cleanliness passed the industrial norm of high-end hospitality sector (<300 RLU) according to Band G of the Hygiene Management Guide issued by 3M.

It is also worth noting that some of the selected areas were pre-occupied meaning that objects inside the room were not removed prior to disinfection. The results of surface cleanliness demonstrated that surface hygiene were greatly improved by the present invention even the locations or areas were occupied.

Where a disinfection is performed by automated means, a stationary or mobile disinfection device as illustrated above can be used. A mobile disinfection device, such as the disinfection robot described herein, is generally more suitable than a stationary device for disinfecting room of larger size. A mobile disinfection device can move around the room reaching each corner of the room while avoiding obstacles such as tables and chairs, and disperse the disinfectant droplets throughout the room in a more consistent manner.

As one skilled in the art will appreciate, no matter a manual, stationary or mobile device is employed for dispersing the disinfectant droplets, it is important to spray sufficient amount of disinfectant to achieve an effective concentration throughout the aircraft cabin (especially at the blocked or less exposed areas) to ensure sufficient coverage and allow sufficient time for the reaction to happen. The treatment time may vary according to the type and quantity of the target microorganism in question, the type of disinfectant used and the pre-treatment conditions of the aircraft cabin.

In some embodiments, to disinfect a confined room of about 200-400 square feet, the effective concentration of the quat-based disinfectant droplets in the room is in the range of 10-40 ppm, and the treatment time is in the range of 20-360 minutes. In some other embodiments, to disinfect a confined room of about 200-400 square feet, the effective concentration of the disinfectant droplets in the room is in the range of 10-40 ppm, and the treatment time is in the range of 20-360 minutes. In some embodiments, to disinfect a confined room of about 1,500-4,000 cubic feet, the effective concentration of the quat-based disinfectant droplets in the room is in the range of 10-40 ppm, and the treatment time is in the range of 20-360 minutes. In some embodiments, to disinfect a confined room of about 1,500-4,000 cubic feet, the effective concentration of the disinfectant droplets in the room is in the range of 10-40 ppm, and the treatment time is in the range of 20-360 minutes.

In one embodiment, there is provided a method of reducing the quantity, cell viability and/or infectivity of a target microorganism in a confined room, the method comprises a step of dispersing into the a confined room a multiplicity of droplets of a disinfectant composition, and a step of allowing a time sufficient for the droplets to distribute throughout the aircraft cabin thereby maintaining an effective concentration of the disinfectant composition in the confined space for a period of time.

In one embodiment, the effective concentration is a concentration of the disinfectant composition in the a confined room that is sufficient to reduce the bioburden of the target microorganism in the confined room by 90.0%-99.9% within a period of time.

In some embodiments, the method described herein reduces the quantity, cell viability and/or infectivity of a target microorganism in a confined room and reduces the bioburden of the target microorganism by 90.0-99.9% within a period of time. In yet other embodiments, the method described herein reduces the RLU reading of a confined room by at least 70%, 75%, 80%, 85%, 90%, or 95% within a period of time.

In one embodiment, the present method of reducing the quantity, cell viability and/or infectivity of a target microorganism in a confined room of about 1,500-4,000 cubic feet comprises dispersing to the room droplets of a disinfectant composition containing the following active ingredients and in accordance with the following parameters:

| | | Con- | | | Average |
|---|---|---|---|---|---|
| | | centration | | | reduction |
| | | of active | | Airborne | of |
| | | ingredient | Aver- | dis- | bacterial |
| | | within | age | infectant | level |
| | Examples of | the | droplet | contact | after |
| Active | disinfectant | room | size | time | treatment |
| ingredient | composition | (ppm) | (μm) | (min) | (%) |
| Hypo- | NANOCYN | 10-40 | 15-40 | 30-240 | 90-99.9 |
| chlorous | | | | | |
| acid | | | | | |
| Quat | ECOLAB | 5-40 | 15-40 | 30-120 | 90-99.9 |
| | DESGUARD | | | | |
| | 20, | | | | |
| | EVICATE | | | | |
| | Pro, | | | | |
| | DIVERSEY | | | | |

In one embodiment, the present method of reducing the quantity, cell viability and/or infectivity of a target microorganism in a confined room of about 4,000 to 10,000 cubic feet comprises dispersing to the room droplets of a disinfectant composition containing the following active ingredients and in accordance with the following parameters:

| | | | | | Average |
|---|---|---|---|---|---|
| | | Con- | | | reduction |
| | | centration | | Airborne | of |
| | | of active | Aver- | dis- | bacterial |
| | | ingredient | age | infectant | level |
| | Examples of | within the | droplet | contact | after |
| Active | disinfectant | room | size | time | treatment |
| ingredient | composition | (ppm) | (μm) | (min) | (%) |
| Hypo- | NANOCYN | 10-40 | 15-40 | 30-240 | 90-99.9 |
| chlorous | | | | | |
| acid | | | | | |
| Quat | ECOLAB | 5-40 | 15-40 | 30-120 | 90-99.9 |
| | DESGUARD | | | | |
| | 20, | | | | |
| | EVICATE | | | | |
| | Pro, | | | | |
| | DIVERSEY | | | | |

### OTHER EMBODIMENTS

In some embodiments of the methods described herein, after the commencement of the step of dispersing the droplets of a disinfectant composition into the confined space, various data such as the concentration of the active ingredient of the disinfectant composition or the quantity of the target microorganism is collected at regular intervals or continuously during the dispersing step. In one embodiment, the concentration of the active ingredient of the disinfectant composition in the confined space is detected at regular intervals or continuously during the dispersing step. The user or the device dispersing the disinfectant droplets may compare such detected concentration to a predetermined target concentration of the active ingredient thereby determining whether the target concentration has been attained in the confined space. In yet another embodiment, the user or the device dispersing the disinfectant droplets may cease the dispersing step when the detected concentration has achieved a value equal to or larger than the predetermined target concentration for a period of time such as 5, 10 or 15 minutes.

The example embodiments may include additional devices and networks beyond those shown. Further, the functionality described as being performed by one device may be distributed and performed by two or more devices. Multiple devices may also be combined into a single device, which may perform the functionality of the combined devices.

The various participants and elements described herein may operate one or more computer apparatuses to facilitate the functions described herein. Any of the elements in the above-described Figures, including any servers, user devices, or databases, may use any suitable number of subsystems to facilitate the functions described herein.

Any of the software components or functions described in this application, may be implemented as software code or computer readable instructions that may be executed by at least one processor using any suitable computer language such as, for example, Java, C++, or Python using, for example, conventional or object-oriented techniques.

The software code may be stored as a series of instructions or commands on a non-transitory computer readable medium, such as a random access memory (RAM), a read only memory (ROM), a magnetic medium such as a hard-drive or a floppy disk, or an optical medium such as a CD-ROM. Any such computer readable medium may reside on or within a single computational apparatus and may be present on or within different computational apparatuses within a system or network.

It may be understood that the present invention as described above may be implemented in the form of control logic using computer software in a modular or integrated manner. Based on the disclosure and teachings provided herein, a person of ordinary skill in the art may know and appreciate other ways and/or methods to implement the present invention using hardware, software, or a combination of hardware and software.

The above description is illustrative and is not restrictive. Many variations of embodiments may become apparent to those skilled in the art upon review of the disclosure. The scope embodiments should, therefore, be determined not with reference to the above description, but instead should be determined with reference to the pending claims along with their full scope or equivalents.

One or more features from any embodiment may be combined with one or more features of any other embodiment without departing from the scope embodiments. A recitation of “a”, “an” or “the” is intended to mean “one or more” unless specifically indicated to the contrary. Recitation of “and/or” is intended to represent the most inclusive sense of the term unless specifically indicated to the contrary.

One or more of the elements of the present system may be claimed as means for accomplishing a particular function. Where such means-plus-function elements are used to describe certain elements of a claimed system it may be understood by those of ordinary skill in the art having the present specification, figures and claims before them, that the corresponding structure includes a computer, processor, or microprocessor (as the case may be) programmed to perform the particularly recited function using functionality found in a computer after special programming and/or by implementing one or more algorithms to achieve the recited functionality as recited in the claims or steps described above. As would be understood by those of ordinary skill in the art that algorithm may be expressed within this disclosure as a mathematical formula, a flow chart, a narrative, and/or in any other manner that provides sufficient structure for those of ordinary skill in the art to implement the recited process and its equivalents.

While the present disclosure may be embodied in many different forms, the drawings and discussion are presented with the understanding that the present disclosure is an exemplification of the principles of one or more inventions and is not intended to limit any one embodiments to the embodiments illustrated.

Further advantages and modifications of the above described system and method may readily occur to those skilled in the art.

The disclosure, in its broader aspects, is therefore not limited to the specific details, representative system and methods, and illustrative examples shown and described above. Various modifications and variations may be made to the above specification without departing from the scope or spirit of the present disclosure, and it is intended that the present disclosure covers all such modifications and variations provided they come within the scope of the following claims and their equivalents.

What is claimed is:

1. A disinfection robot comprising:
a chassis;
at least two wheels, wherein one of the wheels disposed on one side of the chassis and the other wheel disposed on the other side of the chassis;
a disinfection module configured to disperse atomized droplets of disinfectants to a surrounding;
a display; and
a computer system operatively coupled to one or more sensors,
wherein the computer system is configured to receive real-time data from the one or more sensors, generate an output after processing the real-time data using a machine learning algorithm, and optimize the chassis, the disinfection module and the display based on the generated output from the machine learning algorithm.

2. The disinfection robot of claim 1, wherein the display is an liquid crystal display (LCD) touch screen.

3. The disinfection robot of claim 1, wherein the computer system is configured to:
receive, from the one or more sensors, environmental data including at least one of temperature, humidity, and pressure;
process the environmental data using the machine learning algorithm; and
adjust, based on the output from the machine learning algorithm and the environmental data, at least one of:
biocidal efficacy of the atomized droplets of disinfectants, and
an amount of atomized droplets of disinfectants dispersed by the disinfection module.

4. The disinfection robot of claim 1, wherein the computer system is configured to analyze the real-time data using the machine learning algorithm to identify at least one of a material composition of objects to be disinfected and a type of objects to be disinfected, and adjust a disinfection strategy of the disinfection robot based on the identified material composition or type of objects.

5. The disinfection robot of claim 1, wherein the disinfection module comprises:
a fluid tank configured to store at least one type of disinfectant;
a pumping system configured to drive the movement of the disinfectant liquid within the robot;
an atomization system configured to generate the atomized droplets of disinfectant;
a dispenser configured to dispense the atomized droplets of disinfectant from the robot to its surrounding; and
a flow adjustment module configured to control the flow rate and direction of the droplets of disinfectant.

6. The disinfection robot of claim 5, wherein the atomization system comprises one or more atomizing elements configured to convert the disinfectant liquid into the atomized droplets, and wherein the atomization system generates the atomized droplets of disinfectant by ultrasonic means, electrostatic means, or a combination thereof.

7. The disinfection robot of claim 5, wherein the disinfection module further comprising:
a configuration system fluidly connected to the atomization system, wherein the configuration system is configured to receive the atomizing droplets from the atomization system and optimize the size of the atomized droplets of disinfectants dispersed from the disinfection module.

8. The disinfection robot of claim 7, wherein the configuration system comprises one or more block plates configured to block the escape of the atomized droplets beyond a predetermined size from the configuration system.

9. The disinfection robot of claim 8, wherein the one or more block plates comprises a first plate and at least one side plate angled toward the atomization system and away from an outlet of the configuration system.

10. The disinfection robot of claim 9, wherein the atomized droplets dispersed from the disinfection module is in the range of 2 microns to 40 microns in size and at least 95% of the atomized droplets has substantially the same size.

11. The disinfection robot of claim 1, further comprising a detection module, wherein said detection module comprises an molecular testing device configured to detect the presence of microorganism, wherein the molecular testing device is antigen-based or nucleic acid-based.

12. The disinfection robot of claim 1, further comprising an identification module configured to identify and track the disinfectant.

13. The disinfection robot of claim 1, wherein the robot further comprises a handle on one side of the chassis.

14. The disinfection robot of claim 1, further comprising a powering module configured to supply energy for operating the robot, wherein the power powering module comprises at least one changeable battery.

15. The disinfection robot of claim 1, further comprising one or more locking means on one or more sides of the chassis, wherein the one or more locking means are configured to pair with one or more complementary components on one or more straps to allow a user to carry the robot on the body of the user via the one or more straps.

16. The disinfection robot of claim 1, wherein the weight of the robot is in a range of about 10 kg to about 15 kg; and wherein the disinfection robot has a width of no more than 34 cm.

17. The disinfection robot of claim 1, further comprising a communication module configured to connect the robot to a remote computer system or external device, and receive instructions from the remote computer system or external device.

18. The disinfection robot of claim 1, wherein the disinfection robot is an autonomous robot and further comprises:
one or more motors configured to drive the wheels;
at least one navigation sensors configured to receive signals about the surrounding environment; and
where the computer system is configured to receive and process signals from the at least one navigation sensors, and generate one or more commands controlling navigation and operation of the robot.

19. The disinfection robot of claim 18, wherein the robot has a tolerance distance of at least 20 mm on the left side from its center and at least 20 mm on the right side from its center.

20. The disinfection robot of claim 18, the robot is capable of one or more of the following:
turning at the spot in 360 degrees;
steering around tight corners; and
climbing to 15 degrees.
